(19) United States
(12) Patent Application Publication
(10) Pub. No.: US 2025/0041224 A1
(43) Pub. Date: Feb. 6, 2025

(54) DRY POWDER INHALATION DELIVERY OF PHARMACEUTICALS

(71) Applicant: Quench Medical Inc., St. Paul, MN (US)

(72) Inventor: Bryce Calvin BEVERLIN, II, St. Paul, MN (US)

(21) Appl. No.: 18/919,985

(22) Filed: Oct. 18, 2024

Related U.S. Application Data
(63) Continuation of application No. PCT/US2023/019355, filed on Apr. 21, 2023.
(60) Provisional application No. 63/333,750, filed on Apr. 22, 2022.

(51) Int. Cl. A61K 9/14; A61K 9/00; A61K 31/7068; A61P 35/00

(52) U.S. Cl. CPC A61K 9/145; A61K 9/0075; A61K 9/143; A61K 31/7068; A61P 35/00

(57) ABSTRACT

The present disclosure relates to pharmaceutical formulations that include a dry powder including an active pharmaceutical ingredient (API), a growth enhancing excipient, and a dispersion enhancing agent, and methods of treatment and delivery using such pharmaceutical formulations. The pharmaceutical formulation may be a dry powder having a particle size of from about 0.5 μm to about 2.5 μm volume median diameter.

### CROSS-REFERENCE TO PRIORITY APPLICATION

This application is a bypass continuation of International Application No. PCT/US2023/019355, filed Apr. 21, 2023, which claims the benefit of U.S. Provisional Patent Application No. 63/333,750, filed Apr. 22, 2022, the disclosures of each are hereby incorporated by reference as if written herein in their entireties.

### BACKGROUND

### Technical Field

The present disclosure relates to pharmaceutical formulations that include a dry powder including an active pharmaceutical ingredient (API), a growth enhancing excipient, and a dispersion enhancing agent, and methods of treatment using such pharmaceutical formulations.

### Description of the Related Art

Lung cancer is the leading cause of cancer mortality with a 5-year survival rate of less than 20% (Siegel, R., D. Naishadham and A. Jemal, CA Cancer J. Clin., 2012, 62: 10-29). The National Cancer Institute estimates that 235,760 new cases of lung cancer were diagnosed in the United States in 2021 with an estimated 131,880 deaths, which is nearly 22% of all cancer deaths. While detectable and localized tumors are generally treated by surgical resection, more than two-thirds of patients do not qualify for surgery due to difficult-to-access tumors, advanced stage cancer, or other health issues.

Non-small cell lung cancer (NSCLC) is the most common type of lung cancer. Clinically, non-small cell pulmonary tumors typically appear as rounded nodules of variable size, scattered throughout the periphery of both lungs. These are nearly impossible to completely remove with current surgical techniques (Travis, W. D., et al., Proc. Am. Thorac. Soc., 2011. 8:381-385). In addition, most newly diagnosed patients who undergo surgery have undetectable microscopic tumor sites at the time of diagnosis and thus would benefit from preoperative and postoperative chemotherapy. Despite the use of surgery and aggressive combination chemotherapy, the development of microscopic and visible lung tumors continues to be a major challenge in treating this disease.

### BRIEF SUMMARY

In one aspect, the present disclosure provides a pharmaceutical formulation including at least one active pharmaceutical ingredient (API) or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter.

Another embodiment of the present disclosure is a method of treating lung cancer including administering to a lung of a subject in need thereof of a therapeutically effective amount of a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, and wherein the pharmaceutical formulation is administered as a dry powder aerosol.

Another embodiment of the present disclosure is a method of delivery of a chemotherapeutic agent to a lung of a subject, administering a pharmaceutical formulation that includes at least one chemotherapeutic agent; at least one growth enhancing excipient; and at least one dispersion enhancing agent; wherein the pharmaceutical formulation is a dry powder having a particle size of from about 0.5 μm to about 2.5 μm volume median diameter, the pharmaceutical formulation is administered using a dry powder inhaler, and the pharmaceutical formulation is deposited in the lung in an amount of at least about 50% by weight of the pharmaceutical formulation.

Another embodiment of the present disclosure is use of a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, in the manufacture of a medicament for the treatment of lung cancer, wherein the pharmaceutical formulation is to be administered to a lung of a subject as a dry powder aerosol.

Another embodiment of the present disclosure is a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, for use in the treatment of lung cancer in a subject, wherein the pharmaceutical formulation is to be administered to a lung of the subject as a dry powder aerosol.

Another embodiment of the present disclosure is use of the pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, for treating lung cancer in a subject, wherein the pharmaceutical formulation is for administration to a lung of the subject as a dry powder aerosol.

Another embodiment of the present disclosure is a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, wherein the API includes a chemotherapeutic agent. Optionally in this embodiment or any other embodiment disclosed herein, the chemotherapeutic agent may be gemcitabine. Optionally in this embodiment or any other embodiment of the present disclosure, the growth enhancing excipient may include sodium chloride (NaCl), mannitol, or a combination thereof. Optionally in this embodiment or any other embodiment of the present disclosure the dispersion enhancing agent may include leucine. Optionally in this embodiment or any other embodiment of the present disclosure the pharmaceutical formulation may include the API in an amount from about 30% to about 70% by weight of the formulation. Optionally in this embodiment or any other embodiment of the present disclosure the pharmaceutical formulation may include the growth enhancing excipient in an amount from about 15% to about 50% by weight of the formulation.

Optionally in this embodiment or any other embodiment of the present disclosure the pharmaceutical formulation may include the dispersion enhancing agent in an amount from about 15% to about 45% by weight of the formulation. Optionally in this embodiment or any other embodiment of the present disclosure the pharmaceutical formulation may include: the API in an amount of about 50% by weight of the formulation; the growth enhancing excipient in an amount of about 20% by weight of the formulation; and the dispersion enhancing agent in an amount of about 30% by weight of the formulation. Optionally in this embodiment or any other embodiment of the present disclosure the pharmaceutical formulation may include: the API in an amount of about 40% by weight of the formulation; the growth enhancing excipient in an amount of about 40% by weight of the formulation; and the dispersion enhancing agent in an amount of about 20% by weight of the formulation. Optionally in this embodiment or any other embodiment of the present disclosure, the pharmaceutical formulation may include the API in an amount of about 55% by weight of the formulation; the growth enhancing excipient in an amount of about 20% by weight of the formulation; and the dispersion enhancing agent in an amount of about 25% by weight of the formulation. Optionally in this embodiment or any other embodiment of the present disclosure, the pharmaceutical formulation may include: the API in an amount of about 60% by weight of the formulation; the growth enhancing excipient in an amount of about 20% by weight of the formulation; and the dispersion enhancing agent in an amount of about 20% by weight of the formulation. Optionally in this embodiment or any other embodiment of the present disclosure the pharmaceutical formulation may include the API in an amount of about 40% by weight of the formulation; the growth enhancing excipient in an amount of about 20% by weight of the formulation; and the dispersion enhancing agent in an amount of about 40% by weight of the formulation. Optionally in this embodiment or any other embodiment of the present disclosure the particle size may be from about 1 μm to about 2.1 μm volume median diameter. Optionally in this embodiment or any other embodiment of the present disclosure the particle size may be about 1.5 μm volume median diameter. Optionally in this embodiment or any other embodiment of the present disclosure the particle size may be about 1.2 μm volume median diameter. Optionally in this embodiment or any other embodiment of the present disclosure the particle size may be about 1.3 μm volume median diameter. Optionally in this embodiment or any other embodiment of the present disclosure the particle size may be about 1.7 μm volume median diameter. Optionally in this embodiment or any other embodiment of the present disclosure, the particle size may be about 1.9 μm volume median diameter. Optionally in this embodiment or any other embodiment of the present disclosure, a particle size distribution may have a D10 of about 0.6 μm, a D50 of about 1.2 μm, and a D90 of about 2.5 μm. Optionally in this embodiment or any other embodiment of the present disclosure a particle size distribution may have a D10 of about 0.8 μm, a D50 of about 1.5 μm, and a D90 of about 2.8 μm. Optionally in this embodiment or any other embodiment of the present disclosure a particle size distribution may have a D10 of about 0.7 μm, a D50 of about 1.3 μm, and a D90 of about 2.5 μm. Optionally in this embodiment or any other embodiment of the present disclosure a particle size distribution may have a D10 of about 0.8 μm, a D50 of about 1.7 μm, and a D90 of about 3.3 μm. Optionally in this embodiment or any other embodiment of the present disclosure, a particle size distribution may have a D10 of about 0.9 μm, a D50 of about 1.9 μm, and a D90 of about 3.7 μm. Optionally in this embodiment or any other embodiment of the present disclosure, the pharmaceutical formulation may be packaged in a capsule or a blister pack. Optionally in this embodiment or any other embodiment of the present disclosure, the pharmaceutical formulation may be manufactured by spray drying. Optionally in this embodiment or any other embodiment of the present disclosure, the volume median diameter may not change by more than 10% following storage for at least one month at 25° C. (77° F.) and 60% relative humidity. Optionally in this embodiment or any other embodiment of the present disclosure, the volume median diameter may not change by more than 20% following storage for at least one month at 40° C. (104° F.) and 75% relative humidity. Optionally in this embodiment or any other embodiment of the present disclosure, the pharmaceutical formulation may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity. Optionally in this embodiment or any other embodiment of the present disclosure, the pharmaceutical formulation may be formulated for administration by aerosol delivery. Optionally in this embodiment or any other embodiment of the present disclosure, the pharmaceutical formulation may be formulated for administration using a dry powder inhaler. Optionally in this embodiment or any other embodiment of the present disclosure, the pharmaceutical formulation may be packaged into a single capsule for administration using the dry powder inhaler.

Another embodiment of the present disclosure is a method of treating or preventing lung cancer including administering to a lung of a subject in need thereof of a therapeutically effective amount of a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, the pharmaceutical formulation is administered as a dry powder aerosol, and the pharmaceutical formulation is deposited in the lung in an amount of at least about 50% by weight of the pharmaceutical formulation. Optionally in this embodiment or any other embodiment disclosed herein, the pharmaceutical formulation may be deposited in the lung in an amount of at least about 80% by weight of the pharmaceutical formulation. Optionally in this embodiment or any other embodiment of the present disclosure, the pharmaceutical formulation may be deposited in the throat, mouth, or a combination thereof, in an amount of less than about 20% by weight of the pharmaceutical formulation. Optionally in this embodiment or any other embodiment of the present disclosure, the subject may have a suboptimal or inadequate response to intravenous delivery of the APL Optionally in this embodiment or any other embodiment of the present disclosure, administering the pharmaceutical formulation may result in a reduced tumor burden as compared to intravenous delivery of an equivalent dose of the API. Optionally in this embodiment or any other embodiment of the present disclosure, the lung cancer may be a secondary lung cancer.

Another embodiment of the present disclosure is a method of delivery of a chemotherapeutic agent to a lung of a subject, including administering a pharmaceutical formulation that includes at least one chemotherapeutic agent; at least one growth enhancing excipient; and at least one dispersion enhancing agent; wherein the pharmaceutical formulation is a dry powder having a particle size of from about 0.5 μm to about 2.5 μm volume median diameter, the pharmaceutical formulation is administered using a dry powder inhaler, the pharmaceutical formulation is deposited in the lung in an amount of at least about 50% by weight of the pharmaceutical formulation, and wherein the chemotherapeutic agent is gemcitabine, or a pharmaceutically acceptable salt, ester, derivative, analog, prodrug, hydrate, or solvate thereof, or any combination thereof. Optionally in this embodiment or any other embodiment disclosed herein, the growth enhancing excipient may include sodium chloride (NaCl), mannitol, or a combination thereof. Optionally in this embodiment or any other embodiment of the present disclosure, the dispersion enhancing agent may include leucine. Optionally in this embodiment or any other embodiment of the present disclosure, the pharmaceutical formulation may include the chemotherapeutic agent in an amount from about 30% to about 70% by weight of the formulation. Optionally in this embodiment or any other embodiment of the present disclosure, the pharmaceutical formulation may include the growth enhancing excipient in an amount from about 15% to about 50% by weight of the formulation. Optionally in this embodiment or any other embodiment of the present disclosure, the pharmaceutical formulation may include the dispersion enhancing agent in an amount from about 15% to about 45% by weight of the formulation. Optionally in this embodiment or any other embodiment of the present disclosure, the pharmaceutical formulation may include: the chemotherapeutic agent in an amount of about 50% by weight of the formulation; the growth enhancing excipient in an amount of about 20% by weight of the formulation; and the dispersion enhancing agent in an amount of about 30% by weight of the formulation. Optionally in this embodiment or any other embodiment of the present disclosure, the pharmaceutical formulation may include: the chemotherapeutic agent in an amount of about 40% by weight of the formulation; the growth enhancing excipient in an amount of about 40% by weight of the formulation; and the dispersion enhancing agent in an amount of about 20% by weight of the formulation. Optionally in this embodiment or any other embodiment of the present disclosure, the pharmaceutical formulation may include: the chemotherapeutic agent in an amount of about 55% by weight of the formulation; the growth enhancing excipient in an amount of about 20% by weight of the formulation; and the dispersion enhancing agent in an amount of about 25% by weight of the formulation. Optionally in this embodiment or any other embodiment of the present disclosure, the pharmaceutical formulation may include: the chemotherapeutic agent in an amount of about 60% by weight of the formulation; the growth enhancing excipient in an amount of about 20% by weight of the formulation; and the dispersion enhancing agent in an amount of about 20% by weight of the formulation. Optionally in this embodiment or any other embodiment of the present disclosure, the pharmaceutical formulation may include: the chemotherapeutic agent in an amount of about 40% by weight of the formulation; the growth enhancing excipient in an amount of about 20% by weight of the formulation; and the dispersion enhancing agent in an amount of about 40% by weight of the formulation. Optionally in this embodiment or any other embodiment of the present disclosure, the particle size may be from about 1 μm to about 2.1 μm volume median diameter. Optionally in this embodiment or any other embodiment of the present disclosure, the particle size may be about 1.5 μm volume median diameter. Optionally in this embodiment or any other embodiment of the present disclosure, the particle size may be about 1.2 μm volume median diameter. Optionally in this embodiment or any other embodiment of the present disclosure, the particle size may be about 1.3 μm volume median diameter. Optionally in this embodiment or any other embodiment of the present disclosure, the particle size may be about 1.7 μm volume median diameter. Optionally in this embodiment or any other embodiment of the present disclosure, the particle size may be about 1.9 μm volume median diameter. Optionally in this embodiment or any other embodiment of the present disclosure, a particle size distribution may have a D10 of about 0.6 μm, a D50 of about 1.2 μm, and a D90 of about 2.5 μm. Optionally in this embodiment or any other embodiment of the present disclosure, a particle size distribution may have a D10 of about 0.8 μm, a D50 of about 1.5 μm, and a D90 of about 2.8 μm. Optionally in this embodiment or any other embodiment of the present disclosure, a particle size distribution may have a D10 of about 0.7 μm, a D50 of about 1.3 μm, and a D90 of about 2.5 μm. Optionally in this embodiment or any other embodiment of the present disclosure, a particle size distribution may have a D10 of about 0.8 μm, a D50 of about 1.7 μm, and a D90 of about 3.3 μm. Optionally in this embodiment or any other embodiment of the present disclosure, a particle size distribution may have a D10 of about 0.9 μm, a D50 of about 1.9 μm, and a D90 of about 3.7 μm. Optionally in this embodiment or any other embodiment of the present disclosure, the pharmaceutical formulation may be packaged in a capsule or a blister pack. Optionally in this embodiment or any other embodiment of the present disclosure, the pharmaceutical formulation may be manufactured by spray drying. Optionally in this embodiment or any other embodiment of the present disclosure, the volume median diameter of the pharmaceutical formulation may not change by more than 10% following storage for at least one month at 25° C. (77° F.) and 60% relative humidity. Optionally in this embodiment or any other embodiment of the present disclosure, the volume median diameter of the pharmaceutical formulation may not change by more than 20% following storage for at least one month at 40° C. (104° F.) and 75% relative humidity. Optionally in this embodiment or any other embodiment of the present disclosure, the pharmaceutical formulation may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity. Optionally in this embodiment or any other embodiment of the present disclosure, the pharmaceutical formulation may be packaged into a single capsule for administration using the dry powder inhaler.

Another embodiment of the present disclosure is a use of a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, in the manufacture of a medicament for the treatment of lung cancer, wherein the pharmaceutical formulation is to be administered to a lung of a subject as a dry powder aerosol, and wherein the API includes a chemotherapeutic agent. Optionally in this embodiment or any other embodiment disclosed herein, the chemotherapeutic agent may be gemcitabine. Optionally in this embodiment or any other embodiment of the present disclosure, the growth enhancing excipient may include sodium chloride (NaCl), mannitol, or a combination thereof. Optionally in this embodiment or any other embodiment of the present disclosure the dispersion enhancing agent may include leucine. Optionally in this embodiment or any other embodiment of the present disclosure the pharmaceutical formulation may include the API in an amount from about 30% to about 70% by weight of the formulation. Optionally in this embodiment or any other embodiment of the present disclosure the pharmaceutical formulation may include the growth enhancing excipient in an amount from about 15% to about 50% by weight of the formulation.

Optionally in this embodiment or any other embodiment of the present disclosure the pharmaceutical formulation may include the dispersion enhancing agent in an amount from about 15% to about 45% by weight of the formulation. Optionally in this embodiment or any other embodiment of the present disclosure the pharmaceutical formulation may include: the API in an amount of about 50% by weight of the formulation; the growth enhancing excipient in an amount of about 20% by weight of the formulation; and the dispersion enhancing agent in an amount of about 30% by weight of the formulation. Optionally in this embodiment or any other embodiment of the present disclosure the pharmaceutical formulation may include: the API in an amount of about 40% by weight of the formulation; the growth enhancing excipient in an amount of about 40% by weight of the formulation; and the dispersion enhancing agent in an amount of about 20% by weight of the formulation. Optionally in this embodiment or any other embodiment of the present disclosure, the pharmaceutical formulation may include the API in an amount of about 55% by weight of the formulation; the growth enhancing excipient in an amount of about 20% by weight of the formulation; and the dispersion enhancing agent in an amount of about 25% by weight of the formulation. Optionally in this embodiment or any other embodiment of the present disclosure, the pharmaceutical formulation may include: the API in an amount of about 60% by weight of the formulation; the growth enhancing excipient in an amount of about 20% by weight of the formulation; and the dispersion enhancing agent in an amount of about 20% by weight of the formulation. Optionally in this embodiment or any other embodiment of the present disclosure the pharmaceutical formulation may include the API in an amount of about 40% by weight of the formulation; the growth enhancing excipient in an amount of about 20% by weight of the formulation; and the dispersion enhancing agent in an amount of about 40% by weight of the formulation. Optionally in this embodiment or any other embodiment of the present disclosure the particle size may be from about 1 μm to about 2.1 μm volume median diameter. Optionally in this embodiment or any other embodiment of the present disclosure the particle size may be about 1.5 μm volume median diameter. Optionally in this embodiment or any other embodiment of the present disclosure the particle size may be about 1.2 μm volume median diameter. Optionally in this embodiment or any other embodiment of the present disclosure the particle size may be about 1.3 μm volume median diameter. Optionally in this embodiment or any other embodiment of the present disclosure the particle size may be about 1.7 μm volume median diameter. Optionally in this embodiment or any other embodiment of the present disclosure, the particle size may be about 1.9 μm volume median diameter. Optionally in this embodiment or any other embodiment of the present disclosure, a particle size distribution may have a D10 of about 0.6 μm, a D50 of about 1.2 μm, and a D90 of about 2.5 μm. Optionally in this embodiment or any other embodiment of the present disclosure a particle size distribution may have a D10 of about 0.8 μm, a D50 of about 1.5 μm, and a D90 of about 2.8 μm. Optionally in this embodiment or any other embodiment of the present disclosure a particle size distribution may have a D10 of about 0.7 μm, a D50 of about 1.3 μm, and a D90 of about 2.5 μm. Optionally in this embodiment or any other embodiment of the present disclosure a particle size distribution may have a D10 of about 0.8 μm, a D50 of about 1.7 μm, and a D90 of about 3.3 μm. Optionally in this embodiment or any other embodiment of the present disclosure, a particle size distribution may have a D10 of about 0.9 μm, a D50 of about 1.9 μm, and a D90 of about 3.7 μm. Optionally in this embodiment or any other embodiment of the present disclosure, the pharmaceutical formulation may be packaged in a capsule or a blister pack. Optionally in this embodiment or any other embodiment of the present disclosure, the pharmaceutical formulation may be manufactured by spray drying. Optionally in this embodiment or any other embodiment of the present disclosure, the volume median diameter may not change by more than 10% following storage for at least one month at 25° C. (77° F.) and 60% relative humidity. Optionally in this embodiment or any other embodiment of the present disclosure, the volume median diameter may not change by more than 20% following storage for at least one month at 40° C. (104° F.) and 75% relative humidity. Optionally in this embodiment or any other embodiment of the present disclosure, the pharmaceutical formulation may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity. Optionally in this embodiment or any other embodiment of the present disclosure, the pharmaceutical formulation may be formulated for administration by aerosol delivery. Optionally in this embodiment or any other embodiment of the present disclosure, the pharmaceutical formulation may be formulated for administration using a dry powder inhaler. Optionally in this embodiment or any other embodiment of the present disclosure, the pharmaceutical formulation may be packaged into a single capsule for administration using the dry powder inhaler. Optionally in this embodiment or any other embodiment of the present enclosure, the pharmaceutical formulation is deposited in the lung in an amount of at least about 50% by weight of the pharmaceutical formulation. Optionally in this embodiment or any other embodiment disclosed herein, the pharmaceutical formulation may be deposited in the lung in an amount of at least about 80% by weight of the pharmaceutical formulation. Optionally in this embodiment or any other embodiment of the present disclosure, the pharmaceutical formulation may be deposited in the throat, mouth, or a combination thereof, in an amount of less than about 20% by weight of the pharmaceutical formulation. Optionally in this embodiment or any other embodiment of the present disclosure, the subject may have a suboptimal or inadequate response to intravenous delivery of the API. Optionally in this embodiment or any other embodiment of the present disclosure, administering the pharmaceutical formulation may result in a reduced tumor burden as compared to intravenous delivery of an equivalent dose of the API. Optionally in this embodiment or any other embodiment of the present disclosure, the lung cancer may be a secondary lung cancer.

Another embodiment of the present disclosure is a pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, for use in the treatment of lung cancer in a subject, wherein the pharmaceutical formulation is to be administered to a lung of the subject as a dry powder aerosol, wherein the API includes a chemotherapeutic agent. Optionally in this embodiment or any other embodiment disclosed herein, the chemotherapeutic agent may be gemcitabine. Optionally in this embodiment or any other embodiment of the present disclosure, the growth enhancing excipient may include sodium chloride (NaCl), mannitol, or a combination thereof. Optionally in this embodiment or any other embodiment of the present disclosure the dispersion enhancing agent may include leucine. Optionally in this embodiment or any other embodiment of the present disclosure the pharmaceutical formulation may include the API in an amount from about 30% to about 70% by weight of the formulation. Optionally in this embodiment or any other embodiment of the present disclosure the pharmaceutical formulation may include the growth enhancing excipient in an amount from about 15% to about 50% by weight of the formulation. Optionally in this embodiment or any other embodiment of the present disclosure the pharmaceutical formulation may include the dispersion enhancing agent in an amount from about 15% to about 45% by weight of the formulation. Optionally in this embodiment or any other embodiment of the present disclosure the pharmaceutical formulation may include: the API in an amount of about 50% by weight of the formulation; the growth enhancing excipient in an amount of about 20% by weight of the formulation; and the dispersion enhancing agent in an amount of about 30% by weight of the formulation. Optionally in this embodiment or any other embodiment of the present disclosure the pharmaceutical formulation may include: the API in an amount of about 40% by weight of the formulation; the growth enhancing excipient in an amount of about 40% by weight of the formulation; and the dispersion enhancing agent in an amount of about 20% by weight of the formulation. Optionally in this embodiment or any other embodiment of the present disclosure, the pharmaceutical formulation may include the API in an amount of about 55% by weight of the formulation; the growth enhancing excipient in an amount of about 20% by weight of the formulation; and the dispersion enhancing agent in an amount of about 25% by weight of the formulation. Optionally in this embodiment or any other embodiment of the present disclosure, the pharmaceutical formulation may include: the API in an amount of about 60% by weight of the formulation; the growth enhancing excipient in an amount of about 20% by weight of the formulation; and the dispersion enhancing agent in an amount of about 20% by weight of the formulation. Optionally in this embodiment or any other embodiment of the present disclosure the pharmaceutical formulation may include the API in an amount of about 40% by weight of the formulation; the growth enhancing excipient in an amount of about 20% by weight of the formulation; and the dispersion enhancing agent in an amount of about 40% by weight of the formulation. Optionally in this embodiment or any other embodiment of the present disclosure the particle size may be from about 1 μm to about 2.1 μm volume median diameter. Optionally in this embodiment or any other embodiment of the present disclosure the particle size may be about 1.5 μm volume median diameter. Optionally in this embodiment or any other embodiment of the present disclosure the particle size may be about 1.2 μm volume median diameter. Optionally in this embodiment or any other embodiment of the present disclosure the particle size may be about 1.3 μm volume median diameter. Optionally in this embodiment or any other embodiment of the present disclosure the particle size may be about 1.7 μm volume median diameter. Optionally in this embodiment or any other embodiment of the present disclosure, the particle size may be about 1.9 μm volume median diameter. Optionally in this embodiment or any other embodiment of the present disclosure, a particle size distribution may have a D10 of about 0.6 μm, a D50 of about 1.2 μm, and a D90 of about 2.5 μm. Optionally in this embodiment or any other embodiment of the present disclosure a particle size distribution may have a D10 of about 0.8 μm, a D50 of about 1.5 μm, and a D90 of about 2.8 μm. Optionally in this embodiment or any other embodiment of the present disclosure a particle size distribution may have a D10 of about 0.7 μm, a D50 of about 1.3 μm, and a D90 of about 2.5 μm. Optionally in this embodiment or any other embodiment of the present disclosure a particle size distribution may have a D10 of about 0.8 μm, a D50 of about 1.7 μm, and a D90 of about 3.3 μm. Optionally in this embodiment or any other embodiment of the present disclosure, a particle size distribution may have a D10 of about 0.9 μm, a D50 of about 1.9 μm, and a D90 of about 3.7 μm. Optionally in this embodiment or any other embodiment of the present disclosure, the pharmaceutical formulation may be packaged in a capsule or a blister pack. Optionally in this embodiment or any other embodiment of the present disclosure, the pharmaceutical formulation may be manufactured by spray drying. Optionally in this embodiment or any other embodiment of the present disclosure, the volume median diameter may not change by more than 10% following storage for at least one month at 25° C. (77° F.) and 60% relative humidity. Optionally in this embodiment or any other embodiment of the present disclosure, the volume median diameter may not change by more than 20% following storage for at least one month at 40° C. (104° F.) and 75% relative humidity. Optionally in this embodiment or any other embodiment of the present disclosure, the pharmaceutical formulation may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity. Optionally in this embodiment or any other embodiment of the present disclosure, the pharmaceutical formulation may be formulated for administration by aerosol delivery. Optionally in this embodiment or any other embodiment of the present disclosure, the pharmaceutical formulation may be formulated for administration using a dry powder inhaler. Optionally in this embodiment or any other embodiment of the present disclosure, the pharmaceutical formulation may be packaged into a single capsule for administration using the dry powder inhaler. Optionally in this embodiment or any other embodiment of the present enclosure, the pharmaceutical formulation is deposited in the lung in an amount of at least about 50% by weight of the pharmaceutical formulation. Optionally in this embodiment or any other embodiment disclosed herein, the pharmaceutical formulation may be deposited in the lung in an amount of at least about 80% by weight of the pharmaceutical formulation. Optionally in this embodiment or any other embodiment of the present disclosure, the pharmaceutical formulation may be deposited in the throat, mouth, or a combination thereof, in an amount of less than about 20% by weight of the pharmaceutical formulation. Optionally in this embodiment or any other embodiment of the present disclosure, the subject may have a suboptimal or inadequate response to intravenous delivery of the API. Optionally in this embodiment or any other embodiment of the present disclosure, administering the pharmaceutical formulation may result in a reduced tumor burden as compared to intravenous delivery of an equivalent dose of the API. Optionally in this embodiment or any other embodiment of the present disclosure, the lung cancer may be a secondary lung cancer.

Another embodiment of the present disclosure is use of the pharmaceutical formulation including at least one API or a pharmaceutically acceptable salt, ester, derivative, analog, prodrug, hydrate, or solvate thereof, or any combination thereof; at least one growth enhancing excipient; and at least one dispersion enhancing agent; wherein the pharmaceutical formulation is a dry powder having a particle size of from about 0.5 μm to about 2.5 μm volume median diameter, for treating lung cancer in a subject, wherein the pharmaceutical formulation is for administration to a lung of the subject as a dry powder aerosol, wherein the API includes a chemotherapeutic agent. Optionally in this embodiment or any other embodiment disclosed herein, the chemotherapeutic agent may be gemcitabine. Optionally in this embodiment or any other embodiment of the present disclosure, the growth enhancing excipient may include sodium chloride (NaCl), mannitol, or a combination thereof. Optionally in this embodiment or any other embodiment of the present disclosure the dispersion enhancing agent may include leucine. Optionally in this embodiment or any other embodiment of the present disclosure the pharmaceutical formulation may include the API in an amount from about 30% to about 70% by weight of the formulation. Optionally in this embodiment or any other embodiment of the present disclosure the pharmaceutical formulation may include the growth enhancing excipient in an amount from about 15% to about 50% by weight of the formulation. Optionally in this embodiment or any other embodiment of the present disclosure the pharmaceutical formulation may include the dispersion enhancing agent in an amount from about 15% to about 45% by weight of the formulation. Optionally in this embodiment or any other embodiment of the present disclosure the pharmaceutical formulation may include: the API in an amount of about 50% by weight of the formulation; the growth enhancing excipient in an amount of about 20% by weight of the formulation; and the dispersion enhancing agent in an amount of about 30% by weight of the formulation. Optionally in this embodiment or any other embodiment of the present disclosure the pharmaceutical formulation may include: the API in an amount of about 40% by weight of the formulation; the growth enhancing excipient in an amount of about 40% by weight of the formulation; and the dispersion enhancing agent in an amount of about 20% by weight of the formulation. Optionally in this embodiment or any other embodiment of the present disclosure, the pharmaceutical formulation may include the API in an amount of about 55% by weight of the formulation; the growth enhancing excipient in an amount of about 20% by weight of the formulation; and the dispersion enhancing agent in an amount of about 25% by weight of the formulation. Optionally in this embodiment or any other embodiment of the present disclosure, the pharmaceutical formulation may include: the API in an amount of about 60% by weight of the formulation; the growth enhancing excipient in an amount of about 20% by weight of the formulation; and the dispersion enhancing agent in an amount of about 20% by weight of the formulation. Optionally in this embodiment or any other embodiment of the present disclosure the pharmaceutical formulation may include the API in an amount of about 40% by weight of the formulation; the growth enhancing excipient in an amount of about 20% by weight of the formulation; and the dispersion enhancing agent in an amount of about 40% by weight of the formulation. Optionally in this embodiment or any other embodiment of the present disclosure the particle size may be from about 1 μm to about 2.1 μm volume median diameter. Optionally in this embodiment or any other embodiment of the present disclosure the particle size may be about 1.5 μm volume median diameter. Optionally in this embodiment or any other embodiment of the present disclosure the particle size may be about 1.2 μm volume median diameter. Optionally in this embodiment or any other embodiment of the present disclosure the particle size may be about 1.3 μm volume median diameter. Optionally in this embodiment or any other embodiment of the present disclosure the particle size may be about 1.7 μm volume median diameter. Optionally in this embodiment or any other embodiment of the present disclosure, the particle size may be about 1.9 μm volume median diameter. Optionally in this embodiment or any other embodiment of the present disclosure, a particle size distribution may have a D10 of about 0.6 μm, a D50 of about 1.2 μm, and a D90 of about 2.5 μm. Optionally in this embodiment or any other embodiment of the present disclosure a particle size distribution may have a D10 of about 0.8 μm, a D50 of about 1.5 μm, and a D90 of about 2.8 μm. Optionally in this embodiment or any other embodiment of the present disclosure a particle size distribution may have a D10 of about 0.7 μm, a D50 of about 1.3 μm, and a D90 of about 2.5 μm. Optionally in this embodiment or any other embodiment of the present disclosure a particle size distribution may have a D10 of about 0.8 μm, a D50 of about 1.7 μm, and a D90 of about 3.3 μm. Optionally in this embodiment or any other embodiment of the present disclosure, a particle size distribution may have a D10 of about 0.9 μm, a D50 of about 1.9 μm, and a D90 of about 3.7 μm. Optionally in this embodiment or any other embodiment of the present disclosure, the pharmaceutical formulation may be packaged in a capsule or a blister pack. Optionally in this embodiment or any other embodiment of the present disclosure, the pharmaceutical formulation may be manufactured by spray drying. Optionally in this embodiment or any other embodiment of the present disclosure, the volume median diameter may not change by more than 10% following storage for at least one month at 25° C. (77° F.) and 60% relative humidity. Optionally in this embodiment or any other embodiment of the present disclosure, the volume median diameter may not change by more than 20% following storage for at least one month at 40° C. (104° F.) and 75% relative humidity. Optionally in this embodiment or any other embodiment of the present disclosure, the pharmaceutical formulation may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity. Optionally in this embodiment or any other embodiment of the present disclosure, the pharmaceutical formulation may be formulated for administration by aerosol delivery. Optionally in this embodiment or any other embodiment of the present disclosure, the pharmaceutical formulation may be formulated for administration using a dry powder inhaler. Optionally in this embodiment or any other embodiment of the present disclosure, the pharmaceutical formulation may be packaged into a single capsule for administration using the dry powder inhaler. Optionally in this embodiment or any other embodiment of the present enclosure, the pharmaceutical formulation is deposited in the lung in an amount of at least about 50% by weight of the pharmaceutical formulation. Optionally in this embodiment or any other embodiment disclosed herein, the pharmaceutical formulation may be deposited in the lung in an amount of at least about 80% by weight of the pharmaceutical formulation. Optionally in this embodiment or any other embodiment of the present disclosure, the pharmaceutical formulation may be deposited in the throat, mouth, or a combination thereof, in an amount of less than about 20% by weight of the pharmaceutical formulation. Optionally in this embodiment or any other embodiment of the present disclosure, the subject may have a suboptimal or inadequate response to intravenous delivery of the API. Optionally in this embodiment or any other embodiment of the present disclosure, administering the pharmaceutical formulation may result in a reduced tumor burden as compared to intravenous delivery of an equivalent dose of the API. Optionally in this embodiment or any other embodiment of the present disclosure, the lung cancer may be a secondary lung cancer.

### BRIEF DESCRIPTION OF THE SEVERAL VIEWS OF THE DRAWINGS

FIG. 1 shows example compositions of pharmaceutical formulations of the present disclosure.

FIG. 2 shows a graph of particle size distributions of example pharmaceutical formulations.

FIG. 3 shows a graph of initial particle size compared to particle size following storage for one month at 25° C./60% relative humidity and 40° C./75% relative humidity for example pharmaceutical formulations.

FIG. 4 shows a graph of the percent of fine particle fraction (FPF) following storage for one month at ambient conditions (25° C./60% relative humidity) for example pharmaceutical formulations.

FIG. 5 shows a graph of lung weight of Rowett nude rats after treatment once-a-week for four consecutive weeks in the following treatment groups: no cancer (naïve control; circles), untreated (sham control; squares), intravenous (IV) standard of care treatment with 1.0 mg/kg gemcitabine (standard of care—IV; triangles), inhaled dose of an example pharmaceutical formulation of the present disclosure at 1.0 mg/kg gemcitabine (inhalation—IV dose; inverted triangles) and inhaled dose of an example pharmaceutical formulation of the present disclosure at 0.5 mg/kg gemcitabine (inhalation—½ IV dose; diamonds).

FIG. 6 shows hematoxylin and eosin stained lung samples of Rowett nude rats after treatment once-a-week for four consecutive weeks in the following treatment groups: Top row shows results for treatment with an inhaled dose of an example pharmaceutical formulation of the present disclosure at 0.5 mg/kg gemcitabine; bottom row shows results for intravenous (IV) standard of care treatment with 1.0 mg/kg gemcitabine.

### DETAILED DESCRIPTION

The present disclosure relates to pharmaceutical formulations that include a dry powder including an active pharmaceutical ingredient (API), a growth enhancing excipient, and a dispersion enhancing agent, and methods of treatment and delivery using such pharmaceutical formulations.

The present disclosure contemplates methods of treatment or prevention of lung cancer (e.g., non-small cell lung cancer) by aerosol administration of an API (e.g., a chemotherapeutic agent such as gemcitabine or an analog or derivative thereof), and dry powder pharmaceutical formulations including the same. Gemcitabine may be administered by several different routes, including intravenous, intramuscular, subcutaneous, intranasally, and via aerosol. However, when treating a pulmonary process alone, delivery of gemcitabine directly to the lung may provide the benefit of avoiding exposure to other organ systems and reducing potential toxicity. The methods disclosed herein contemplate the use of devices designed for pulmonary delivery of therapeutic products, including dry powder inhalers.

More specifically, the present disclosure provides a pharmaceutical formulation including at least one API, or a pharmaceutically acceptable salt, ester, derivative, analog, prodrug, hydrate, or solvate thereof, or any combination thereof, at least one growth enhancing excipient, and at least one dispersion enhancing agent, wherein the pharmaceutical formulation is a dry powder having a particle size of from about 0.5 μm to about 2.5 μm volume median diameter. The present disclosure also provides a method of treating or preventing lung cancer including aerosol delivery to a lung of a subject in need thereof of a therapeutically effective amount of a pharmaceutical formulation. The present disclosure further provides a method of delivery of a chemotherapeutic agent to a lung of a subject including using a dry powder inhaler to administer a pharmaceutical formulation including at least one chemotherapeutic agent, at least one growth enhancing excipient, and at least one dispersion enhancing agent, wherein the pharmaceutical formulation is a dry powder having a particle size of from about 0.5 μm to about 2.5 μm volume median diameter, and wherein the pharmaceutical formulation is deposited in the lung in an amount of at least about 50% by weight of the pharmaceutical formulation.

Among the many advantages of the pharmaceutical formulations and methods of the present disclosure, only some of which are alluded to herein, the pharmaceutical formulations and methods of the present disclosure may, among other benefits, provide for improved efficacy, safety, decreased toxicity and/or increased patient comfort for treatment of lung cancer. In some embodiments, the pharmaceutical formulations and methods of the present disclosure may provide an increased efficacy as compared to certain other pharmaceutical formulations and delivery methods, at least in part due to an increased local concentration of the API in the lung. In some embodiments, the pharmaceutical formulations of the present disclosure are delivered at higher efficiencies to the lung, while nearly completely avoiding deposition in the mouth and throat, as compared to certain other pharmaceutical formulations. In some embodiments, this may increase the local concentration of API in the tumor tissue. In some embodiments, the pharmaceutical formulations of the present disclosure may provide improved efficacy compared to standard of care intravenous formulations at matched and reduced doses.

In some embodiments, the pharmaceutical formulations of the present disclosure may provide an increased safety as compared to certain other pharmaceutical formulations and delivery methods, at least in part due to decreased systemic levels of the API in circulation, which may result in decreased systemic toxicity. In certain embodiments, lower mouth-throat deposition may avoid off-target side effects of toxic active pharmaceutical ingredients, and/or may minimize wasted active pharmaceutical ingredient. Further, targeted delivery of inhaled pharmaceutical formulations of the present disclosure directly to the lung may reduce an effective dose, which may further reduce side effects. In some embodiments, the pharmaceutical formulations of the present disclosure may provide improved storage stability as compared to certain other pharmaceutical formulations. In certain embodiments, the pharmaceutical formulations and methods of the present disclosure may provide for increased patient comfort, at least in part due the ability of administration to be in non-clinical settings, needle-free, painless, and completed in less than five minutes.

In contrast to systemic delivery of chemotherapy (e.g., a chemotherapeutic drug is delivered via the bloodstream to all parts of the body), inhalation may deliver a chemotherapeutic drug directly to tumor tissues in the lung. This may enhance efficacy and safety due to increased local drug concentration in the lung, decreased systemic drug levels in the circulation, and decreased systemic toxicity (Koshkina, N. V., et al., Cancer Chemother. Pharmacol., 1999. 44:187-192). Given the potential metabolism of chemotherapeutic drugs such as gemcitabine in cancer cells, treatment with gemcitabine by inhalation may be highly beneficial as it may increase local drug concentration in the tumor tissue compared to systemic drug delivery methods. Inhalation may further decrease systemic drug levels to reduce systemic toxicity effects, which is especially important with chemotherapeutic cytotoxic drugs. Inhalation may have major benefits including increased local effects by avoiding first pass metabolism, may be administered in non-clinical settings, may increase patient comfort towards treatment by being needle-free, painless, and may be delivered in less than five minutes. In vivo efficacy and safety has been demonstrated in treating primary tumors with ad-hoc nebulized gemcitabine aerosols in animals and humans (Zarogoulidis, P., et al., Transl. Lung Cancer Res., 2013. 2:E17-22; Lemarie, E., et al., J Aerosol. Med. Pulm. Drug Deliv., 2011. 24:261-70). In addition, using 1/10th the systemic dose, in vivo efficacy and safety has been demonstrated in treating secondary metastatic tumors with nebulized gemcitabine and has also shown potential to eliminate local recurrence (Gordon, N. and E. S. Kleinerman, J. Aerosol Med. Pulm. Drug Deliv., 2010. 23: 189-96; Koshkina, N. V. and E. S. Kleinerman, Int. J. Cancer, 2005. 116:458-63). Disadvantages of nebulization may include low peripheral lung deposition, high off-target drug wasted in the mouth and throat, lengthy treatment duration, equipment contamination, and toxic aerosol risk to medical staff. Given the strong potential for improved treatment, a highly efficient and safer chemotherapeutic inhalation alternative is desirable. There is further a need to develop new ways of treating smaller lung cancer sites with targeted and less invasive therapies as earlier detection of lung tumors becomes more common.

In addition to effective treatment of tumors in the lung, inhaled gemcitabine has been shown to prevent metastatic spread, with little evidence of toxicity to normal tissues. The efficacy of aerosol delivery in the dog and baboon has been demonstrated with gemcitabine as well as other chemotherapeutic and immunomodulatory agents (Khanna, C., et al., Clin. Cancer Res., 1996, 2:721-734; Rodriguez, C. O., et al., J. Aerosol Med Pulm. Drug Deliv., 2010. 23:197-206; Hershey, A. E., et al., Clin. Cancer Res., 1999, 5:2653-2659). Aerosol gemcitabine has also shown feasibility and safety in a clinical setting, however, side effects observed in these studies are nearly all attributable to nebulization as the method of delivery, making a non-nebulized formulation preferable (Lemarie, E., et al., J. Aerosol Med. Pulm. Drug Deliv., 2011, 24:261-270; Gagnadoux, F., et al., J. Aerosol Med. Pulm. Drug Deliv., 2008, 21:61-70).

Gemcitabine is an FDA approved chemotherapeutic agent and recommended as an evidence-based Level 1 regimen for use in treating non-small cell lung cancer (NSCLC) patients according to current National Comprehensive Cancer Network (NCCN) Guidelines. It may be used as a first-line therapy for advanced metastatic adenocarcinoma and squamous cell carcinoma, where it is recommended be used in combination with docetaxel, vinorelbine, carboplatin, or cisplatin, or as a stand-alone maintenance therapy. Additionally, there are extensive and promising in vivo studies that demonstrate aerosolized gemcitabine may be a highly effective treatment as a stand-alone therapy or in combination with others, such as cisplatin.

The present disclosure relates to a new type of targeted lung cancer treatment with a highly efficient and novel dry powder pharmaceutical formulation that may include gemcitabine. The pharmaceutical formulation may be optimized to maximize efficacy with minimal dose and minimal toxicity. This may significantly improve treatment for patients with non-small cell lung cancer. The pharmaceutical formulation of the present disclosure may be a dry powder that may utilize, at least in part, for example, excipient enhanced growth (EEG) technology for delivery. Excipient enhanced growth (EEG) is a methodology that may significantly outperform current inhaler technologies and pioneer the field of inhaled chemotherapies (PW, L., et al., Respiratory Drug Delivery 2012: Davis Healthcare International; River Grove, IL. 2012, 61-69; Longest, P. W., et al., Beyond Small Particles: Development of DPIs that Target Small Airways and Alveoli, in Respiratory Drug Delivery. 2014).

Controlling the particle size of the pharmaceutical formulations of the present disclosure may avoid mouth-throat deposition and target deposition within the lung. Delivery of medicines with current nebulized, dry powder, and pressurized metered dose devices, producing particles above approximately 5 μm in diameter, have very high mouth-throat depositional loss, very low lung deposition and extremely low peripheral airway deposition (Newman, S. P. and W. W. Busse, Respir. Med., 2002, 96:293-304; Longest, P. W., et al., Pharm. Res., 2012. 29:1670-1688; Delvadia, R. R., P. W. Longest, and P. R. Byron, J. Aerosol Med. Pulm. Drug Deliv., 2012, 25:32-40; Lipworth, B., A. Manoharan, and W. Anderson, Lancet Respir. Med., 2014, 2:497-506; Dolovich, M. B. and J. P. Mitchell, Can. Respir. J., 2004, 11:489-495). Particles between 2 μm and 5 μm may have less mouth-throat and improved lung deposition, however peripheral airway deposition may still be low, especially using nebulizers (Lipworth, B., A. Manoharan, and W. Anderson, Lancet Respir. Med., 2014, 2:497-506; Dolovich, M. B. and J. P. Mitchell, Can. Respir. J., 2004, 11:489-495; Laube, B. L., et al., Eur. Respir. J., 2011. 37:1308-1331). Current nebulized and dry powder inhaler (DPI) devices may have up to 70% losses in the mouth-throat (Newman, S. P. and W. W. Busse, Respir. Med., 2002, 96:293-304; Longest, P. W., et al., Pharm. Res., 2012. 29:1670-1688; Delvadia, R. R., P. W. Longest, and P. R. Byron, J. Aerosol Med. Pulm. Drug Deliv., 2012, 25:32-40). Cytotoxic chemotherapy drugs may pose an even greater risk from off-target deposit in the mouth-throat. Therefore, there remains a need to efficiently deliver chemotherapy medication to the lung, especially the peripheral airways, with minimal mouth-throat loss, and to treat patients with pulmonary tumors using a convenient dry powder inhaler, which may also minimize healthcare worker exposure.

As described above, delivery of smaller aerosol particles to the patient may minimize mouth-throat deposition of a dry powder pharmaceutical formulation. However, reducing the size of the particles alone may not increase deposition, as the particles may lack the inertia to deposit in the lungs and a large fraction may be exhaled (Hinds, W., Aerosol Technology: Properties, Behavior, and Measurement of Airborne Particles, 1999, Wiley Interscience). The pharmaceutical formulations of the present disclosure may include micrometer-sized particles that increase in size within the airways using the resident humidity of the lungs (e.g., particles based on excipient enhanced growth (EEG) technology). This may achieve an optimal size for targeted deposition and may result in increased deposition of the pharmaceutical formulation in the lung. In turn, this may result a reduction in variability of dose delivery and an increased consistency of treatment across patient populations.

Without wishing to be bound by theory, optimizing the ratio of an API to excipients may deliver a pharmaceutical formulation to the lung with increased efficiency (see Tian, G., et al., J. Aerosol Med. Pulm. Drug Deliv., 2013. 26:248-265). Certain formulations have been shown to achieve emitted doses greater than 75%, fine particle fractions (fraction <5 μm in size) of greater than 90% and initial mass median aerodynamic diameters less than 1.5 μm, which may result in mouth-throat depositional losses of less than 5%. Current inhaler formulations deliver very little medication into the lungs, typically less than 30%, with less than 1% being deposited in the peripheral airways. In contrast, the formulations described herein may provide delivery at efficiencies of greater than 80% while nearly completely avoiding deposition in the mouth and throat (see, for example, Behara, S. R., et al., Pharm Res, 2014. 31: 360-372). Very low mouth-throat deposition (e.g., <5%) may avoid creating associated off-target side effects of toxic drugs and will minimize wasted drug (see, for example, Behara, S. R., et al., J. Pharm. Sci., 2014. 103: 465-477). Targeted delivery of efficiently inhaled pharmaceutical formulations of the present disclosure directly to the lung may reduce effective doses, which may further reduce side effects. In some embodiments, the pharmaceutical formulations of the present disclosure may provide delivery of gemcitabine, a potent and toxic yet effective chemotherapy drug, directly to lung tumors in targeted doses.

### Definitions

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, size range or integer range is to be understood to include the value of any integer within the recited range and, when appropriate, fractions thereof (such as one tenth and one hundredth of an integer), unless otherwise indicated. Also, any number range recited herein relating to any physical feature, such as particle size or diameter, are to be understood to include any integer within the recited range, unless otherwise indicated. As used herein, the term “about” means ±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

The term “aerosol” as used herein refers to any preparation of a fine mist of particles, including non-liquid particles, e.g., dry powders.

An “aerosol generator” refers to a device used for producing aerosol particles. Examples of aerosol generators include dry powder inhalers, nebulizers, or metered-dose inhalers. A “dry powder inhaler” (DPI) is an aerosol device that delivers the drug in a powdered form, typically with a breath-actuated dosing system.

The term “dry powder” as used herein refers to a composition that contains finely dispersed respirable dry particles that are capable of being dispersed in an inhalation device and subsequently inhaled by a subject. Such a dry powder or dry particle may contain up to about 25%, up to about 20%, or up to about 15% water or other solvent, or be substantially free of water or other solvent, or be anhydrous.

The term “dry particles” as used herein refers to respirable particles that may contain up to about 15% water or other solvent, or be substantially free of water or other solvent, or be anhydrous.

The term “respirable” as used herein refers to particles or powders that are suitable for delivery to the respiratory tract (e.g., pulmonary delivery) in a subject by inhalation.

As used herein, the term “respiratory tract” includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli). The terms “upper airway” and “lower airway” are referred to as “conducting airways.”

As used herein, the terms “administer,” “administration,” or “administering” of an API (e.g., gemcitabine or an analog or derivative thereof), pharmaceutical formulation or composition refers to introducing (e.g., “delivering”) a dry powder and/or dry particles including the API, pharmaceutical formulation or composition to the respiratory tract of a subject.

The term “pharmaceutically acceptable” refers to molecular entities and compositions that do not produce allergic or other serious adverse reactions when administered to a subject using routes well-known in the art.

The term “pharmaceutically acceptable excipient” as used herein means that the excipient can be taken into the lungs with no significant adverse toxicological effects on the lungs. Such excipients are generally regarded as safe (GRAS) by the U.S. Food and Drug Administration.

As used herein, a “subject,” may be any organism capable of developing lung cancer, such as humans, pets, livestock, show animals, zoo specimens, or other animals. For example, a subject may be a human, a non-human primate, dog, cat, rabbit, rat, mouse, guinea pig, horse, cow, sheep, goat, pig, or the like. Subjects in need of administration of pharmaceutical formulations as described herein include subjects at high risk for developing lung cancer as well as subjects presenting with existing lung cancer. Subjects suffering from or suspected of having lung cancer may be identified using methods as described herein and known in the art.

A “subject in need” refers to a subject at high risk of, or suffering from, a disease, disorder or condition that is amenable to treatment or amelioration with a pharmaceutical formulation, compound or a composition thereof provided herein. In certain embodiments, a subject in need is a human.

“Treatment,” “treating” or “ameliorating” refers to medical management of a disease, disorder, or condition of a subject (e.g., a patient), which may be therapeutic, prophylactic/preventative, or a combination treatment thereof. A treatment may improve or decrease the severity at least one symptom of lung cancer, delay worsening or progression of lung cancer, or delay or prevent onset of additional associated diseases. “Reducing the risk of developing lung cancer” refers to preventing or delaying onset of lung cancer or reoccurrence of one or more symptoms of lung cancer.

A “therapeutically effective amount (or dose)” or “effective amount (or dose)” of a compound, composition, or pharmaceutical formulation refers to that amount of compound, composition, or pharmaceutical formulation sufficient to result in amelioration of one or more symptoms of the disease being treated in a statistically significant manner. In the case of a compound, composition, or pharmaceutical formulation administered by inhalation, a therapeutically effective amount may be the amount of a compound, composition, or pharmaceutical formulation present in an aerosolizable composition needed to provide a desired level of a compound, composition, or pharmaceutical formulation at the site of action (e.g., lungs) of a subject to be treated to provide an anticipated physiological, biophysical, biochemical, or pharmacological response. The precise amount may depend upon numerous factors, e.g., the API, the activity of the compound, composition, or pharmaceutical formulation, the delivery device employed, the physical characteristics of the compound, composition, or pharmaceutical formulation, the intended patient use (e.g., the number of doses administered per day), patient considerations, or the like, and can readily be determined by one of ordinary skill in the art. When referring to an individual API administered alone, a therapeutically effective dose refers to that ingredient alone. When referring to a combination, a therapeutically effective dose refers to combined amounts of the active ingredients that result in the therapeutic effect, whether administered serially or simultaneously

A therapeutic effect may include, directly or indirectly, the reduction of one or more symptoms of a lung cancer. A therapeutic effect may also include, directly or indirectly, the arrest, reduction, or elimination of a lung cancer tumor. For example, a therapeutic effect may include a reduction of tumor burden in a subject. In some embodiments, a therapeutic effect may include, directly or indirectly, the reduction of one or more symptoms of a secondary lung cancer (e.g., a cancer that originated from non-lung tissue and has metastasized to the lung). In some embodiments, the therapeutic effect may include, directly or indirectly, arrest, reduction, or elimination of a lung cancer tumor that is a secondary tumor (e.g., a metastatic tumor that originated from non-lung tissue and has metastasized to the lung). Examples of cancers that may metastasize to the lung include, but are not limited to, breast, colon, rectum, head and neck, kidney, osteosarcoma, testicular and uterine cancers as well as lymphomas.

“Emitted Dose” or “ED” provides an indication of the delivery of a drug formulation from a suitable inhaler device after a firing or dispersion event. The ED may be an experimentally determined parameter, and is typically determined using an in vitro device set up, which mimics patient dosing. The ED may be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopia convention, Rockville, Md., 13^{th} Revision, 222-225, 2007. For dry powder inhaler dosage forms, the ED may corresponds to the percentage of active pharmaceutical ingredient (e.g., gemcitabine, or a pharmaceutically acceptable salt, ester, derivative, analog, prodrug, hydrate, or solvate thereof) drawn from a dosage form and that exits the mouthpiece of an inhaler device.

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” An analog or derivative may have different chemical, biological or physical properties from the parent compound, such as being more hydrophilic or having altered reactivity. Derivatization (e.g., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiquitination, esterification, and amidation.

The term “derivative” also refers to all solvates, for example, hydrates or adducts (e.g., adducts with alcohols), active metabolites, and salts of a parent compound. The type of salt may depend on the nature of the moieties within the compound. For example, acidic groups, such as carboxylic acid groups, may form alkali metal salts or alkaline earth metal salts (e.g., sodium salts, potassium salts, magnesium salts, calcium salts, and also salts with physiologically tolerable quaternary ammonium ions and acid addition salts with ammonia and physiologically tolerable organic amines such as, for example, triethylamine, ethanolamine or tris-(2-hydroxyethyl) amine). Basic groups may form acid addition salts with, for example, inorganic acids such as hydrochloric acid, sulfuric acid or phosphoric acid, or with organic carboxylic acids or sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, may be present as zwitterions. Salts may be obtained by customary methods known to those skilled in the art, for example, by combining a compound with an inorganic or organic acid or base in a solvent or diluent, or from other salts by cation exchange or anion exchange.

### Formulations

The pharmaceutical formulations of the present disclosure may include at least one API or a pharmaceutically acceptable salt, ester, derivative, analog, prodrug, hydrate, or solvate thereof, or any combination thereof, at least one growth enhancing excipient; and at least one dispersion enhancing agent. In some embodiments, the pharmaceutical formulation is a dry powder. In some embodiments, the pharmaceutical formulation has a particle size of from about 0.5 μm to about 2.5 μm volume median diameter.

In some embodiments, the API may be a chemotherapeutic agent. Suitable chemotherapeutic agents include, but are not limited to, gemcitabine. In some embodiments, the API comprises or consists of gemcitabine. Gemcitabine is a is a nucleoside analog in which the hydrogen atoms on the 2′ carbon of deoxycytidine are replaced by fluorine atoms. In addition, gemcitabine may refer to a composition having the following structure (I), or a pharmaceutically acceptable salt, ester, derivative, analog, prodrug, hydrate, or solvate thereof:

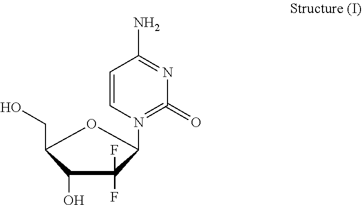

Gemcitabine may be chemically synthesized from, for example, enantiopure D-glyceraldehyde (R)-2, using methods known in the art.

The pharmaceutical formulations of the present disclosure may include the API (e.g., gemcitabine) in an amount from about 30% to about 70% by weight of the formulation. In some embodiments, the pharmaceutical formulation may include the API in an amount from about 35% to about 65%, from about 40% to about 65%, from about 40% to about 60%, from about 45% to about 60%, or from about 50% to about 60%, all by weight of the formulation. In some embodiments, the pharmaceutical formulation may include the API in an amount of about 30%, 35%, 40%, 45%, 50%, 55%, 60%, 65%, or 70% by weight of the formulation.

In some embodiments, the growth enhancing excipient may be any excipient that is hydroscopic. Suitable growth enhancing excipients include, but are not limited to, salts such as NaCl, KCl, zinc chloride, calcium chloride, magnesium chloride, potassium carbonate, potassium phosphate, carnallite, ferric ammonium citrate, magnesium sulfate, sodium sulfite, calcium oxide, ammonium sulfate; sugars such as mannitol, sorbitol, glucose, maltose, galactose, fructose, sucrose, glycols such as polyethylene glycols, propylene glycol, glycerol; organic acids such as citric acid, sulfuric acid, malonic acid, adipic acid; lactams such as 2-pyrrolidone, polyvinylpyrrolidone (PVP); or any combination thereof. The pharmaceutical formulation of the present disclosure may include a single growth enhancing excipient, or more than one growth enhancing excipient (e.g., a combination of at least two growth enhancing excipients). In some embodiments, the growth enhancing excipient may include sodium chloride (NaCl), mannitol, or a combination thereof.

The pharmaceutical formulations of the present disclosure may include the growth enhancing excipient in an amount from about 15% to about 50% by weight of the formulation. In some embodiments, the pharmaceutical formulation may include the growth enhancing excipient in an amount from about 15% to about 45%, from about 15% to about 40%, from about 20% to about 40%, from about 15% to about 35%, or from about 25% to about 40%, all by weight of the formulation. In some embodiments, the pharmaceutical formulation may include the growth enhancing excipient in an amount of about 15%, 20%, 25%, 30%, 35%, 40%, 45%, or 50% by weight of the formulation.

In some embodiments, the dispersion enhancing agent may include any excipient that improves the features of the pharmaceutical formulation, for example, by improving the handling characteristics of dry powders (e.g., dispersion, flowability and/or consistency) and/or facilitating manufacturing and filling of unit dosage forms. Suitable dispersion enhancing agents may include, but are not limited to, amino acids (e.g., leucine), peptides, proteins, non-biological polymers, biological polymers, carbohydrates, such as sugars, derivatized sugars such as alditols, aldonic acids, esterified sugars, sugar polymers, or any combination thereof. The pharmaceutical formulation of the present disclosure may include a single dispersion enhancing agent, or more than one dispersion enhancing agent (e.g., a combination of at least two dispersion enhancing agents). In some embodiments, the dispersion enhancing agent may include leucine (e.g., L-leucine).

The pharmaceutical formulations of the present disclosure may include the dispersion enhancing agent in an amount from about 15% to about 45% by weight of the formulation. In some embodiments, the pharmaceutical formulation may include the dispersion enhancing agent in an amount from about 15% to about 40%, from about 15% to about 35%, from about 20% to about 35%, from about 20% to about 30%, or from about 20% to about 25%, all by weight of the formulation. In some embodiments, the pharmaceutical formulation may include the dispersion enhancing agent in an amount of about 15%, 20%, 25%, 30%, 35%, 40%, or 45% by weight of the formulation.

In some embodiments, the pharmaceutical formulation may include the API in an amount of about 50% by weight of the formulation, the growth enhancing excipient in an amount of about 20% by weight of the formulation; and the dispersion enhancing agent in an amount of about 30% by weight of the formulation.

In some embodiments, the pharmaceutical formulation may include the API in an amount of about 40% by weight of the formulation, the growth enhancing excipient in an amount of about 40% by weight of the formulation; and the dispersion enhancing agent in an amount of about 20% by weight of the formulation.

In some embodiments, the pharmaceutical formulation may include the API in an amount of about 55% by weight of the formulation; the growth enhancing excipient in an amount of about 20% by weight of the formulation; and the dispersion enhancing agent in an amount of about 25% by weight of the formulation.

In some embodiments, the pharmaceutical formulation may include the API in an amount of about 60% by weight of the formulation, the growth enhancing excipient in an amount of about 20% by weight of the formulation; and the dispersion enhancing agent in an amount of about 20% by weight of the formulation.

In some embodiments, the pharmaceutical formulation may include the API in an amount of about 40% by weight of the formulation, the growth enhancing excipient in an amount of about 20% by weight of the formulation; and the dispersion enhancing agent in an amount of about 40% by weight of the formulation.

In some embodiments, the pharmaceutical formulation comprises gemcitabine in an amount of about 50% by weight of the formulation; NaCl, mannitol, or combination thereof in an amount of about 20% by weight of the formulation; and leucine in an amount of about 30% by weight of the formulation.

In some embodiments, the pharmaceutical formulation comprises gemcitabine in an amount of about 40% by weight of the formulation; NaCl, mannitol, or combination thereof in an amount of about 40% by weight of the formulation; and leucine in an amount of about 20% by weight of the formulation.

In some embodiments, the pharmaceutical formulation comprises gemcitabine in an amount of about 55% by weight of the formulation; NaCl, mannitol, or combination thereof in an amount of about 20% by weight of the formulation; and leucine in an amount of about 25% by weight of the formulation.

In some embodiments, the pharmaceutical formulation comprises gemcitabine in an amount of about 60% by weight of the formulation; NaCl, mannitol, or combination thereof in an amount of about 20% by weight of the formulation; and leucine in an amount of about 20% by weight of the formulation.

In some embodiments, the pharmaceutical formulation comprises gemcitabine in an amount of about 40% by weight of the formulation, NaCl, mannitol, or combination thereof in an amount of about 20% by weight of the formulation; and leucine in an amount of about 40% by weight of the formulation.

In some embodiments, the pharmaceutical formulation disclosed herein is a dry powder formulation. Without wishing to be bound by theory, it is thought that particles with an aerodynamic diameter of about 0.5 μm to about 3 μm may be delivered to the lung small airways (deep lung). Larger aerodynamic diameters, for example, from about 3 μm to about 5 μm may be delivered to the central and upper airways.

The size of the dry particles may be expressed in a variety of ways that are conventional in the art, such as, fine particle fraction (FPF), volume median diameter (VMD), or mass median aerodynamic diameter (MMAD).

In some embodiments, the pharmaceutical formulations of the present disclosure may be a dry powder having a particle size, for example, between about 0.5 μm and about 5 μm volume median diameter, between about 0.5 μm and about 2.5 μm volume median diameter, or between about 1 μm and about 2.1 μm volume median diameter. The particle size may be, for example, about 0.5 μm, about 0.8 μm, about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 2.1 μm, about 2.2 μm, about 2.3 μm, about 2.4 μm, about 2.5 μm, about 3 μm, about 4 μm, or about 5 μm volume median diameter.

In some embodiments, the particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different therapeutic agent may be administered to target different regions of the lung in one administration.

The particle size distribution of the pharmaceutical formulations of the present disclosure may be described by D10, D50 and D90 values. These parameters refer to the cumulative size distribution, which describes how many percentages of particles in the pharmaceutical formulation are below a certain size. D10, D50, or D90 may be defined as the size value corresponding to cumulative size distribution at 10%, 50%, or 90%, respectively, which represents the size of particles below which 10%, 50%, or 90%, respectively, of the pharmaceutical formulation lies. In some embodiments, the pharmaceutical formulations of the present disclosure may be a dry powder having a particle size distribution with a D10 of from about 0.3 μm to about 1.1 μm. In some embodiments, the pharmaceutical formulations of the present disclosure may be a dry powder having a particle size distribution with a D50 of from about 1 μm to about 2.1 μm. In some embodiments, the pharmaceutical formulations of the present disclosure may be a dry powder having a particle size distribution with a D90 of from about 2 μm to about 4 μm.

In some embodiments, the pharmaceutical formulations of the present disclosure may be a dry powder having a particle size distribution with a D10 of about 0.6 μm, a D50 of about 1.2 μm, and a D90 of about 2.5 μm. In some embodiments, the particle size distribution may have a D10 of about 0.8 μm, a D50 of about 1.5 μm, and a D90 of about 2.8 μm. In some embodiments, the particle size distribution may have a D10 of about 0.7 μm, a D50 of about 1.3 μm, and a D90 of about 2.5 μm. In some embodiments, the particle size distribution may have a D10 of about 0.8 μm, a D50 of about 1.7 μm, and a D90 of about 3.3 μm. In some embodiments, the particle size distribution may have a D10 of about 0.9 μm, a D50 of about 1.9 μm, and a D90 of about 3.7 μm.

Aerodynamic diameter may be determined using time of flight (TOF) measurements. For example, an instrument such as the Model 3225 Aerosizer DSP Particle Size Analyzer (Amherst Process Instrument, Inc., Amherst, Mass.) may be used to measure aerodynamic diameter. The Aerosizer measures the time taken for individual respirable dry particles to pass between two fixed laser beams.

Aerodynamic diameter may also be experimentally determined directly using conventional gravitational settling methods, in which the time required for a sample of respirable dry particles to settle a certain distance is measured. Indirect methods for measuring the mass median aerodynamic diameter include the Andersen Cascade Impactor (ACI) and the multi-stage liquid impinger (MSLI) methods. The methods and instruments for measuring particle aerodynamic diameter are well known in the art.

A MSLI is a device that may be used to measure fine particle fraction. The MLSI operates on the same principles as the ACI, although instead of eight stages, MSLI has five. Additionally, each MSLI stage consists of an ethanol-wetted glass frit instead of a solid plate. The wetted stage is used to prevent particle bounce and re-entrainment, which may occur when using the ACI.

“Mass median aerodynamic diameter” or “MMAD” is a measure of the aerodynamic size of a dispersed particle. The aerodynamic diameter may be used to describe an aerosolized particle in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by inertial impaction, using for example, a New Generation Impactor or NGI available from TSI Instruments, LTD, United Kingdom. (See, e.g., Allen T. Particle size, shape, and distribution. In: Scarlett B, ed. Particle Size Measurement. 4th ed. New York, N.Y.: Chapman and Hall Inc; 1990:124-189).

In some embodiments, respirable dry particles may have an MMAD of about 10 μm or less, such as an MMAD of about 0.5 μm to about 10 μm. In certain embodiments, dry particles may have an MMAD of about 5 μm or less (e.g., about 0.5 μm to about 5 μm, about 1 μm to about 5 μm), about 4 μm or less (e.g., about 0.5 μm to about 4 μm), about 3.8 μm or less (e.g., about 0.5 μm to about 3.8 μm), about 3.5 μm or less (e.g., about 0.5 μm to about 3.5 μm), about 3.2 μm or less (e.g., about 0.5 μm to about 3.2 μm), about 3 μm or less (e.g., about 0.5 μm to about 3.0 μm), about 2.8 μm or less (e.g., about 0.5 μm to about 2.8 μm), about 2.5 μm or less (e.g., about 0.5 μm to about 2.5 μm), about 2.1 μm or less (e.g., about 0.5 μm to about 2.1 μm, or about 1 μm to about 2.1 μm), about 2.0 μm or less (e.g., about 0.5 μm to about 2.0 μm), about 1.9 μm or less (e.g., about 0.5 μm to about 1.9 μm), about 1.8 μm or less (e.g., about 0.5 μm to about 1.8 μm), about 1.7 μm or less (e.g., about 0.5 μm to about 1.7 μm), about 1.6 μm or less (e.g., about 0.5 μm to about 1.6 μm), about 1.5 μm or less (e.g., about 0.5 μm to about 1.5 μm), about 1.4 μm or less (e.g., about 0.5 μm to about 1.4 μm), about 1.3 μm or less (e.g., about 0.5 μm to about 1.3 μm), about 1.2 μm or less (e.g., about 0.5 μm to about 1.2 μm), or about 1.1 μm or less (e.g., about 0.5 μm to about 1.1 μm).

As used herein, the term “dispersible” describes the characteristic of a dry powder or dry particles to be dispelled into a respirable aerosol. Dispersibility of a dry powder or dry particles may be expressed herein as the quotient of the volume median diameter (VMD) measured at a dispersion (i.e., regulator) pressure of 1 bar divided by the VMD measured at a dispersion (i.e., regulator) pressure of 4 bar, or VMD at 0.5 bar divided by the VMD at 4 bar as measured by HELOS/RODOS. These quotients are referred to herein as “¼ bar,” and “0.5/4 bar,” respectively, and dispersibility correlates with a low quotient. For example, ¼ bar refers to the VMD of respirable dry particles or powders emitted from the orifice of a RODOS dry powder disperser (or equivalent technique) at about 1 bar, as measured by a HELOS or other laser diffraction system, divided the VMD of the same respirable dry particles or powders measured at 4 bar by HELOS/RODOS. Thus, a highly dispersible dry powder or dry particles will have a ¼ bar or 0.5/4 bar ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by the subject. Dispersibility may also be assessed by measuring the size emitted from an inhaler as a function of flowrate.

In some embodiments, the dry particles have a volume median diameter (e.g., as measured by HELOS/RODOS at 1.0 bar) of about 10 μm or less (e.g., about 0.1 μm to about 10 μm). In certain embodiments, the dry particles have a volume median diameter of about 9 μm or less (e.g., about 0.1 μm to about 9 μm), about 8 μm or less (e.g., about 0.1 μm to about 8 μm), about 7 μm or less (e.g., about 0.1 μm to about 7 μm), about 6 μm or less (e.g., about 0.1 μm to about 6 μm), about 5 μm or less (e.g., less than 5 μm, about 0.1 μm to about 5 μm), about 4 μm or less (e.g., 0.1 μm to about 4 μm), about 3 μm or less (e.g., 0.1 μm to about 3 μm), about 2 μm or less (e.g., 0.1 μm to about 2 μm), about 1 μm or less (e.g., 0.1 μm to about 1 μm), about 0.5 μm to about 6 μm, about 0.5 μm to about 5 μm, about 0.5 μm to about 4 μm, about 0.5 μm to about 3 μm, about 0.5 μm to about 2.5 μm, about 1 μm to about 2.1 μm, or about 0.5 μm to about 2 μm (e.g., as measured by HELOS/RODOS at 1.0 bar).

In some embodiments, the dry particles are dispersible, and have ¼ bar and/or 0.5/4 bar of about 2.2 or less (e.g., about 1.0 to about 2.2) or about 2.0 or less (e.g., about 1.0 to about 2.0).

In certain embodiments, the dry particles have ¼ bar and/or 0.5/4 bar of about 1.9 or less (e.g., about 1.0 to about 1.9), about 1.8 or less (e.g., about 1.0 to about 1.8), about 1.7 or less (e.g., about 1.0 to about 1.7), about 1.6 or less (e.g., about 1.0 to about 1.6), about 1.5 or less (e.g., about 1.0 to about 1.5), about 1.4 or less (e.g., about 1.0 to about 1.4), about 1.3 or less (e.g., less than 1.3, about 1.0 to about 1.3), about 1.2 or less (e.g., 1.0 to about 1.2), about 1.1 or less (e.g., 1.0 to about 1.1) or the dry particles have ¼ bar of about 1.0.

“Fine particle fraction” or “FPF” may be defined as the mass percent of particles having an aerodynamic diameter of less than 3.5 μm, indicated by FPF<3.5. The particle size analysis may be determined by compendial procedures (USP 26-NF 21. Chapter 601—Physical tests and determinations: aerosols. United States Pharmacopeia. Rockville, Md.: United States Pharmacopeial Convention; 2003:2105-2123; European Pharmacopeia. Section 2.9.18—Preparations for inhalation: aerodynamic assessment of fine particles. European Pharmacopeia. 3rd ed. [Suppl 2001]. Strasbourg, France: Council of Europe; 2002:113-124). The measurement is typically undertaken using a multistage cascade impactor equipped with United States Pharmacopeia/European Pharmacopeia (USP/EP) induction port. This technique provides a direct link with the mass of therapeutically API and particle aerodynamic size, which is accepted as an indication of the likely deposition location within the respiratory tract (Rudolph et al., J Aerosol Sci. 21:306-406, 1990).

Fine particle fraction may be used as one way to characterize the aerosol performance of a dispersed powder. Fine particle fraction describes the size distribution of airborne respirable dry particles. Gravimetric analysis, using a Cascade impactor, is one method of measuring the size distribution, or fine particle fraction, of airborne respirable dry particles. The Andersen Cascade Impactor (ACI) is an eight-stage impactor that can separate aerosols into nine distinct fractions based on aerodynamic size. The size cutoffs of each stage are dependent upon the flow rate at which the ACI is operated. The ACI is made up of multiple stages consisting of a series of nozzles (i.e., a jet plate) and an impaction surface (i.e., an impaction disc). At each stage an aerosol stream passes through the nozzles and impinges upon the surface. Respirable dry particles in the aerosol stream with a large enough inertia will impact upon the plate. Smaller respirable dry particles that do not have enough inertia to impact on the plate will remain in the aerosol stream and be carried to the next stage. Each successive stage of the ACI has a higher aerosol velocity in the nozzles so that smaller respirable dry particles can be collected at each successive stage.

If desired, a two-stage collapsed ACI may also be used to measure fine particle fraction. The two-stage collapsed ACI consists of only the top two stages of the eight-stage ACI and allows for the collection of two separate powder fractions. Specifically, a two-stage collapsed ACI may be calibrated so that the fraction of powder that is collected on stage one is composed of respirable dry particles that have an aerodynamic diameter of less than, e.g., 5.6 μm and greater than 3.5 μm. The fraction of powder passing stage one and depositing on a collection filter is thus composed of respirable dry particles having an aerodynamic diameter of less than 3.5 μm. The airflow at such a calibration is approximately 60 L/min.

An ACI may be used to approximate the emitted dose, which herein is called gravimetric recovered dose and analytical recovered dose. “Gravimetric recovered dose” is defined as the ratio of the powder weighed on all stage filters of the ACI to the nominal dose. “Analytical recovered dose” is defined as the ratio of the powder recovered from rinsing all stages, all stage filters, and the induction port of the ACI to the nominal dose. The FPF_TD (<5.0) is the ratio of the interpolated amount of powder depositing below 5.0 μm on the ACI to the nominal dose. The FPF_RD (<5.0) is the ratio of the interpolated amount of powder depositing below 5.0 μm on the ACI to either the gravimetric recovered dose or the analytical recovered dose.

The terms “FPF (<5.6),” “FPF (<5.6 μm),” and “fine particle fraction of less than 5.6 μm” as used herein, refer to the fraction of a sample of dry particles that have an aerodynamic diameter of less than 5.6 μm. For example, FPF (<5.6) can be determined by dividing the mass of respirable dry particles deposited on the stage one and on the collection filter of a two-stage collapsed Andersen Cascade Impactor (ACI) by the mass of respirable dry particles weighed into a capsule for delivery to the instrument. This parameter may also be identified as “FPF_TD (<5.6),” where TD means total dose. A similar measurement may be conducted using an eight-stage ACI. The eight-stage ACI cutoffs are different at the standard 60 L/min flowrate, but the FPF_TD (<5.6) may be extrapolated from the eight-stage complete data set. The eight-stage ACI result may also be calculated by the USP method of using the dose collected in the ACI instead of what was in the capsule to determine FPF.

The terms “FPF (<5.0),” “FPF (<5.0 μm),” and “fine particle fraction of less than 5.0 μm” as used herein, refer to the fraction of a mass of respirable dry particles that have an aerodynamic diameter of less than 5.0 μm. For example, FPF (<5.0) can be determined by using an eight-stage ACI at the standard 60 L/min flowrate by extrapolating from the eight-stage complete data set. This parameter may also be identified as “FPF_TD (<5.0),” where TD means total dose.

The terms “FPF (<3.5),” “FPF (<3.5 μm),” and “fine particle fraction of less than 3.5 μm” as used herein, refer to the fraction of a mass of respirable dry particles that have an aerodynamic diameter of less than 3.5 μm. For example, FPF (<3.5) can be determined by dividing the mass of respirable dry particles deposited on the collection filter of a two-stage collapsed ACI by the total mass of respirable dry particles weighed into a capsule for delivery to the instrument. This parameter may also be identified as “FPF_TD (<3.5),” where TD means total dose. A similar measurement can be conducted using an eight-stage ACI. The eight-stage ACI result can also be calculated by the USP method of using the dose collected in the ACI instead of what was in the capsule to determine FPF.

The FPF (<5.6) has been demonstrated to correlate to the fraction of the powder that is able to make it into the lung of the patient, while the FPF (<3.5) has been demonstrated to correlate to the fraction of the powder that reaches the deep lung of a patient. These correlations provide a quantitative indicator that can be used for particle optimization.

In some embodiments, the respirable dry powders and dry particles may have an FPF of less than about 5.6 μm (FPF<5.6 μm) of at least about 20%, at least about 30%, at least about 40%, at least about 45%, at least about 50%, at least about 55%, at least about 60%, at least about 65%, or at least about 70%.

In some embodiments, the dry powders and dry particles may have a FPF of less than 5.0 μm (FPF_TD (<5.0 μm)) of at least about 20%, at least about 30%, at least about 45%, at least about 40%, at least about 45%, at least about 50%, at least about 60%, at least about 65% or at least about 70%. In certain embodiments, the dry powders and dry particles may have a FPF of less than 5.0 μm of the emitted dose (FPF_ED (<5.0 μm)) of at least about 45%, at least about 50%, at least about 60%, at least about 65%, at least about 70%, at least about 75%, at least about 80%, or at least about 85%. In certain embodiments, the dry powders and dry particles may have an FPF of less than about 3.5 μm (FPF<3.5 μm) of at least about 20%, at least about 25%, at least about 30%, at least about 35%, at least about 40%, at least about 45%, at least about 50%, at least about 60%, at least about 65%, at least about 70%, at least about 75%, at least about 80%, or at least about 85%.

In some embodiments, the volume median diameter of the particles of the pharmaceutical formulation may not change by more than 10% following storage for at least one month at about 25° C. (77° F.) and about 60% relative humidity. In some embodiments, the volume median diameter of the particles of the pharmaceutical formulation may not change by more than 20% following storage for at least one month at about 40° C. (104° F.) and about 75% relative humidity.

In certain embodiments, the pharmaceutical formulations of the present disclosure may be formulated such that at least 60% of the dry powders and/or dry particles have an FPF of less than about 3.5 μm (FPF<3.5 μm) when the pharmaceutical formulation is administered using a dry powder inhaler following storage for at least one month at 25° C. (77° F.) and 60% relative humidity. In some embodiments, the pharmaceutical formulations of the present disclosure may be formulated such that the particle size of at least 60% of particles is less than about 3.5 μm volume median diameter when the pharmaceutical formulation is administered using a dry powder inhaler following storage for at least one month at 25° C. (77° F.) and 60% relative humidity.

Tap density is a measure of the envelope mass density characterizing a particle. The envelope mass density of a particle of a statistically isotropic shape is defined as the mass of the particle divided by the minimum sphere envelope volume within which it can be enclosed. Features which can contribute to low tap density include irregular surface texture and porous structure. Tap density may be measured using instruments known to those skilled in the art such as the Dual Platform Microprocessor Controlled Tap Density Tester (Vankel, N.C.), a GeoPyc™ instrument (Micrometrics Instrument Corp., Norcross, Ga.), or SOTAX Tap Density Tester model TD2 (SOTAX Corp., Horsham, Pa.). Tap density may be determined using the method of USP Bulk Density and Tapped Density, United States Pharmacopia convention, Rockville, Md., 10^{th} Supplement, 4950-4951, 1999.

The tap density of particles of a dry powder may be obtained by the standard USP tap density measurement. As described above, tap density is a standard measure of the envelope mass density. The envelope mass density of an isotropic particle is defined as the mass of the particle divided by the minimum sphere envelope volume in which it can be enclosed. Features contributing to low tap density may include irregular surface texture and porous structure.

In some embodiments, the pharmaceutical formulations described herein may include a dry powder having a tap density of about 0.1 g/cm³ to about 1.0 g/cm³. For example, the pharmaceutical formulation may include a dry powder with a tap density of about 0.1 g/cm³ to about 0.9 g/cm³, about 0.2 g/cm³ to about 0.9 g/cm³, about 0.2 g/cm³ to about 0.9 g/cm³, about 0.3 g/cm³ to about 0.9 g/cm³, about 0.4 g/cm³ to about 0.9 g/cm³, about 0.5 g/cm³ to about 0.9 g/cm³, or about 0.5 g/cm³ to about 0.8 g/cm³, greater than about 0.4 g/cc, greater than about 0.5 g/cc, greater than about 0.6 g/cc, greater than about 0.7 g/cc, about 0.1 g/cm³ to about 0.8 g/cm³, about 0.1 g/cm³ to about 0.7 g/cm³, about 0.1 g/cm³ to about 0.6 g/cm³, about 0.1 g/cm³ to about 0.5 g/cm³, about 0.1 g/cm³ to about 0.4 g/cm³, about 0.1 g/cm³ to about 0.3 g/cm³, less than 0.3 g/cm³. In certain embodiments, the tap density of the dry powder may be greater than about 0.4 g/cc. In some embodiments, tap density of the dry powder may be greater than about 0.5 g/cc. Alternatively, in certain embodiments tap density of the dry powder may be less than about 0.4 g/cc.

In some embodiments, the pharmaceutical formulations described herein may have a water or solvent content of less than about 15% by weight of the pharmaceutical formulation. For example, the pharmaceutical formulation may have a water or solvent content of less than about 15% by weight, less than about 13% by weight, less than about 11.5% by weight, less than about 10% by weight, less than about 9% by weight, less than about 8% by weight, less than about 7% by weight, less than about 6% by weight, less than about 5% by weight, less than about 4% by weight, less than about 3% by weight, less than about 2% by weight, less than about 1% by weight, less than about 0.75% by weight, less than about 0.5% by weight, less than about 0.25% by weight, or be anhydrous. The pharmaceutical formulation may have a water or solvent content of less than about 6% and greater than about 0.25%, less than about 5.5% and greater than about 0.25%, less than about 5% and greater than about 0.25%, 0.5%, about 0.75%, about 1%, about 2.5%, about 3%, about 3.5%, about 4%, or about 4.5%.

If desired, the pharmaceutical formulation described herein may include a physiologically or pharmaceutically acceptable carrier or excipient. For example, a pharmaceutically acceptable excipient may include any of the standard carbohydrate, sugar alcohol, and amino acid carriers that are known in the art to be useful excipients for inhalation therapy, either alone or in any desired combination. These excipients are generally relatively free-flowing particulates, do not thicken or polymerize upon contact with water, are toxicologically innocuous when inhaled as a dispersed powder and do not significantly interact with the active agent in a manner that adversely affects the desired physiological action of the salts. Carbohydrate excipients that may be useful in this regard include the mono- and polysaccharides. Representative monosaccharides include carbohydrate excipients such as dextrose (anhydrous and the monohydrate; also referred to as glucose and glucose monohydrate), galactose, mannitol, D-mannose, sorbose and the like. Representative disaccharides include lactose, maltose, sucrose, trehalose and the like. Representative trisaccharides include raffinose and the like. Other carbohydrate excipients include maltodextrin and cyclodextrins, such as 2-hydroxypropyl-beta-cyclodextrin can be used as desired. Representative sugar alcohols include mannitol, sorbitol or the like.

Suitable amino acid excipients may include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cystine, glycine, glutamine, serine, threonine, and tyrosine. Representative examples of polar, positively charged amino acids include arginine, histidine and lysine. Representative examples of negatively charged amino acids include aspartic acid and glutamic acid. These amino acids are generally available from commercial sources that provide pharmaceutical grade. In some embodiments, the pharmaceutical formulation may include a dispersion enhancing agent that includes an amino acid. In some embodiments, the dispersion enhancing agent may include leucine. Illustrative pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

### Uses and Methods of Treatment

In some embodiments, the present disclosure provides a method of treating lung cancer comprising administering to a lung of a subject in need thereof a therapeutically effective amount of a pharmaceutical formulation as described herein as a dry powder aerosol. In some embodiments, the present disclosure provides a use of a pharmaceutical formulation as described herein in a method of treating or preventing lung cancer comprising administering to a lung of a subject in need thereof a therapeutically effective amount of a pharmaceutical formulation as described herein as a dry powder aerosol. In certain embodiments, the pharmaceutical formulation is formulated for administration using a dry powder inhaler. In some embodiments, the pharmaceutical formulation may be a dry powder having a particle size of from about 0.5 μm to about 2.5 μm volume median diameter. In some embodiments, the pharmaceutical formulation may be deposited in the lung in an amount of at least about 50% by weight of the pharmaceutical formulation. In some embodiments, the pharmaceutical formulation may be deposited in the lung in an amount of at least about 80% by weight of the pharmaceutical formulation. In some embodiments, the pharmaceutical formulation may be deposited in the throat, mouth, or a combination thereof, in an amount of less than about 20% by weight of the pharmaceutical formulation. In some embodiments, the subject may have a suboptimal or inadequate response to intravenous delivery of an API in the pharmaceutical formulation. In some embodiments, aerosol delivery of the pharmaceutical formulation may result in a reduced tumor burden as compared to intravenous delivery of an equivalent dose of the API. In some embodiments, aerosol delivery of the pharmaceutical formulation may result in a reduced side effects as compared to intravenous delivery of an equivalent dose of the API. Examples of side effects include bone marrow suppression, liver and kidney problems, nausea, fever, rash, shortness of breath, mouth sores, diarrhea, neuropathy, hair loss, or any combination thereof. In some embodiments, the API is gemcitabine.

In some embodiments, the present disclosure provides a method of treating lung cancer, wherein the lung cancer is a secondary lung cancer (e.g., a cancer that originated from non-lung tissue and has metastasized to the lung). In some embodiments, administering to a lung of a subject in need thereof a therapeutically effective amount of a pharmaceutical formulation as described herein may, directly or indirectly, arrest, reduce, or eliminate a lung cancer tumor that is a secondary tumor (e.g., a metastatic tumor that originated from non-lung tissue and has metastasized to the lung). Examples of cancers that may metastasize to the lung include, but are not limited to, breast, colon, rectum, head and neck, kidney, osteosarcoma, testicular, and uterine cancers, as well as lymphomas.

In some embodiments, the pharmaceutical formulations of the present disclosure may be administered to a subject as an adjuvant therapy to surgery and/or radiation therapy (e.g., either before, after, or both before and after the surgery and/or radiation). In some embodiments, the present disclosure provides a method of delivery of a chemotherapeutic agent to a lung of a subject, including using a dry powder inhaler to administer a pharmaceutical formulation. In some embodiments, the pharmaceutical formulation may include at least one chemotherapeutic agent, at least one growth enhancing excipient, and at least one dispersion enhancing agent. In some embodiments, the pharmaceutical formulation may be a dry powder having a particle size of from about 0.5 μm to about 2.5 μm volume median diameter. In some embodiments, the pharmaceutical formulation may be deposited in the lung in an amount of at least about 50% by weight of the pharmaceutical formulation. In some embodiments, the chemotherapeutic agent is gemcitabine.

As described herein, the pharmaceutical formulations of the present disclosure may be administered to a subject by inhalation using a dry powder inhaler. Such dry powder inhalers typically administer the API as a free-flowing powder that is dispersed in a subject's air-stream during inhalation. There are three basic mechanisms of deposition of particles: impaction, sedimentation, and Brownian motion (see, for example, J. M. Padfield. 1987. In: D. Ganderton & T. Jones eds. Drug Delivery to the Respiratory Tract, Ellis Harwood, Chicherster, U.K.). Impaction may occur when particles are unable to stay within the air stream, particularly at airway branches. They may be adsorbed onto the mucus layer covering bronchial walls and cleaned out by mucociliary action. Impaction mostly occurs with particles over 5 μm in diameter. Smaller particles (e.g., smaller than 5 μm) may stay within the air stream and may be transported deep into the lungs. Sedimentation may often occur in the lower respiratory system where airflow is slower. Very small particles (e.g., smaller than 0.6 μm) may deposit by Brownian motion. Deposition by Brownian motion may be undesirable because of difficulty targeting the particles to the alveoli (see, for example, N. Worakul & J. R. Robinson. 2002. In: Polymeric Biomaterials, 2^{nd} ed. S. Dumitriu ed. Marcel Dekker. New York).

The dry powder pharmaceutical formulation of the present disclosure may be administered to the respiratory tract of a subject in need thereof using any suitable method, such as instillation techniques, and/or an inhalation device, such as a dry powder inhaler (DPI). The pharmaceutical formulations of the present disclosure may be administered using the devices and methods described in U.S. Pat. Nos. 8,479,728; 9,433,588; and 10,105,500, incorporated herein by reference. Other dry powder inhaler devices known in the art may be suitable for use with the currently disclosed pharmaceutical formulations and methods. Additional examples of dry powder inhalers include the inhalers disclosed is U.S. Pat. Nos. 4,995,385 and 4,069,819, SPINHALER® (Fisons, Loughborough, U.K.), ROTAHALERS®, DISKHALER®, and DISKUS® (GlaxoSmithKline, Research Triangle Technology Park, North Carolina; see, e.g., U.S. Pat. Nos. 4,353,365; 6,378,519 and 5,035,237, respectively), FLOWCAPSS® (Hovione, Loures, Portugal), INHALATORS® and HANDIHALER® (Boehringer-Ingelheim, Germany), AEROLIZER® (Novartis, Switzerland), TURBUHALER® (AstraZeneca, Wilmington, Del.; see, e.g., U.S. Pat. No. 4,524,769), incorporated herein by reference. Further examples of suitable dry powder inhaler devices are described in U.S. Pat. Nos. 5,415,162; 5,239,993; and 5,715,810, incorporated herein by reference.

Generally, inhalation devices (e.g., dry powder inhalers) may deliver a maximum amount of dry powder or dry particles in a single inhalation, which is related to the capacity of blisters, capsules (e.g., size 000, 00, 0E, 0, 1, 2, 3, and 4, with respective volumetric capacities of 1.37 ml, 950 μl, 770 μl, 680 μl, 480 μl, 360 μl, 270 μl, and 200 μl) or other cartridges that contain the dry particles or dry powder within the inhaler. Alternatively, a desired dose or effective amount may be delivered in two or more inhalations. In some embodiments, each dose that is administered to a subject in need thereof may include an effective amount of respirable dry particles or dry powder. In certain other embodiments, each dose that is administered in an effective amount of respirable dry particles or dry powder may be delivered using no more than about 4 inhalations. For example, each dose of respirable dry particles or dry powder may be administered in a single inhalation, or 2, 3, or 4 inhalations. In some embodiments, the respirable dry particles and dry powder may be administered in a single, breath-activated step using a breath-activated dry powder inhaler. When this type of device is used, the energy of the subject's inhalation may both disperse the respirable dry particles or dry powder and draw them into the respiratory tract.

A pharmaceutical formulation for use in a dry powder inhaler may include an API (e.g., gemcitabine, or a pharmaceutically acceptable salt, ester, derivative, analog, prodrug, hydrate, or solvate thereof, or any combination thereof) in an amount from about 30% to about 70% by weight of the formulation. A pharmaceutical formulation for use in a dry powder inhaler may include a growth enhancing excipient in an amount from about 15% to about 50% by weight of the formulation. A pharmaceutical formulation for use in a dry powder inhaler may include a dispersion enhancing agent in an amount from about 15% to about 45% by weight of the formulation.

The dose of an API (e.g., gemcitabine) that produces a specified effect in 50% of the population may be the median effective dose, abbreviated ED₅₀. In preclinical studies of drugs, the median lethal dose, as determined in experimental animals, is abbreviated as the LD₅₀. The ratio of the LD₅₀ to the ED₅₀ may be an indication of the therapeutic index, which is a statement of how selective the API is in producing the desired versus its adverse effects. Pharmaceutical formulations including the API (e.g., gemcitabine, or a pharmaceutically acceptable salt, ester, derivative, analog, prodrug, hydrate, or solvate thereof, or any combination thereof) that exhibit high therapeutic indices are preferred.

An API (e.g., gemcitabine, or a pharmaceutically acceptable salt, ester, derivative, analog, prodrug, hydrate, or solvate thereof, or any combination thereof), as well as pharmaceutical formulations thereof, may be used to treat or reduce the risk of developing lung cancer, and may be administered to a subject who has or is at risk of developing lung cancer at a therapeutically effective amount or dose. Such a dose may be determined or adjusted depending on various factors, including the specific API or pharmaceutical formulation, the route of administration, the subject's condition, that is, stage of the disease, severity of symptoms caused by the disease, general health status, as well as age, gender, and weight, and other factors apparent to a person skilled in the medical art. Similarly, the dose of the API for treating a disease or disorder may be determined according to parameters understood by a person skilled in the medical art. When referring to a combination, a therapeutically effective dose refers to combined amounts of the APIs that result in the therapeutic effect, whether administered serially or simultaneously (in the same formulation or concurrently in separate formulations). Optimal doses may generally be determined using experimental models and/or clinical trials. Design and execution of studies of an API (including when administered for prophylactic benefit) described herein are well within the skill of a person skilled in the relevant art.

In some embodiments, for a single dose of an API (e.g., gemcitabine, or a pharmaceutically acceptable salt, ester, derivative, analog, prodrug, hydrate, or solvate thereof, or any combination thereof), the amount to be administered to a subject may be in the range from about 0.01-1.5 mg/kg, from about 0.05-1 mg/kg, from about 0.05-0.5 mg/kg, from about 0.05-0.2 mg/kg, from about 0.3-1 mg/kg, or from about 0.5-1 mg/kg, based on the recipient. In some embodiments, for a single dose of the API (e.g., gemcitabine, or a pharmaceutically acceptable salt, ester, derivative, analog, prodrug, hydrate, or solvate thereof, or any combination thereof), the amount to be administered to a subject may be about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1 mg/kg, 1.05 mg/kg or 1.1 mg/kg, based on the recipient.

Depending upon the dosage amount and precise condition to be treated, administration may be over the course of a single day, several days, weeks, months, and years, and may even be for the life of the patient. Illustrative dosing regimens may last a period of at least about a day, a week, from about 1-4 weeks, from about 1-3 months, from about 1-6 months, from about 1-50 weeks, from about 1-12 months, or longer. Depending upon the dosage amount, formulation, and precise condition to be treated, an average interval between treatments may be about 3 hours to about 7 days. In some cases, a suitable average interval between treatments may be about 3 hours to about 12 hours, about 3 hours to about 3 days, or about 12 hours to about 2 days. In certain embodiments, a once-daily (q.d.) administration regimen may be suitable. In some embodiments, a twice-daily (b.i.d.) administration regimen may be suitable. In certain embodiments, a three-times daily (t.i.d.) administration regimen may be suitable.

In still further embodiments, pharmaceutical formulations described herein may be administered with one or more secondary or additional therapeutic agents. The additional therapeutic agents may be administered by any suitable route, such as orally, parenterally (e.g., intravenous, intraarterial, intramuscular, or subcutaneous injection), topically, by inhalation (e.g., intrabronchial, intranasal or oral inhalation, intranasal drops), rectally, vaginally, or any other method of administration known in the art. The pharmaceutical formulations of the present disclosure may be administered before, substantially concurrently with, or subsequent to administration of the other therapeutic agent. The pharmaceutical formulations of the present disclosure and the other therapeutic agent may be administered so as to provide substantial overlap of their pharmacologic activities.

Secondary or additional therapeutic agents may include immunotherapies, chemotherapies, other targeted agents, or any combination thereof. Exemplary secondary or additional therapeutic agents may include, but are not limited to, cisplatin, carboplatin, paclitaxel, albumin-bound paclitaxel, docetaxel, vinorelbine, etoposide, pemetrexed, alectinib, brigatinib, ceritinib, crizotinib, dabrafenib, trametinib, vemurafenib, erlotinib, gefitinib, Osimertinib, bevacizumab, ramucirumab, cetuximab, capmatinib, crizotinib, entrectinib, Larotrectinib, cabozantinib, pralsetinib, selpercatinib, vandetanib, lorlatinib, atezolizumab, nivolumab, ipilimumab, pembrolizumab, or any combination thereof.

In some embodiments, the pharmaceutical formulation may be manufactured by spray drying. In some embodiments, the pharmaceutical formulation may be made, for example, by combining at least one growth enhancing excipient (e.g., sodium chloride (NaCl), mannitol, or a combination thereof) and at least one dispersion enhancing agent (e.g., leucine) with at least one API (e.g., gemcitabine) or a pharmaceutically acceptable salt, ester, derivative, analog, prodrug, hydrate, or solvate thereof, or any combination thereof and then spray drying the components to form a dry powder. The pharmaceutical formulation may be loaded into a dry powder dispenser, or into inhalation cartridges or capsules for use with a dry powder delivery device. In some embodiments, the pharmaceutical formulation is packaged in a capsule or a blister pack. In certain embodiments, the pharmaceutical formulation is packaged into a single capsule for administration using a dry powder inhaler.

### EXAMPLES

The following examples demonstrate laboratory tests conducted to evaluate the properties and in-vivo efficacy of example pharmaceutical formulations according to some embodiments of the present disclosure.

### Example 1

### Development of Gemcitabine Pharmaceutical Formulations

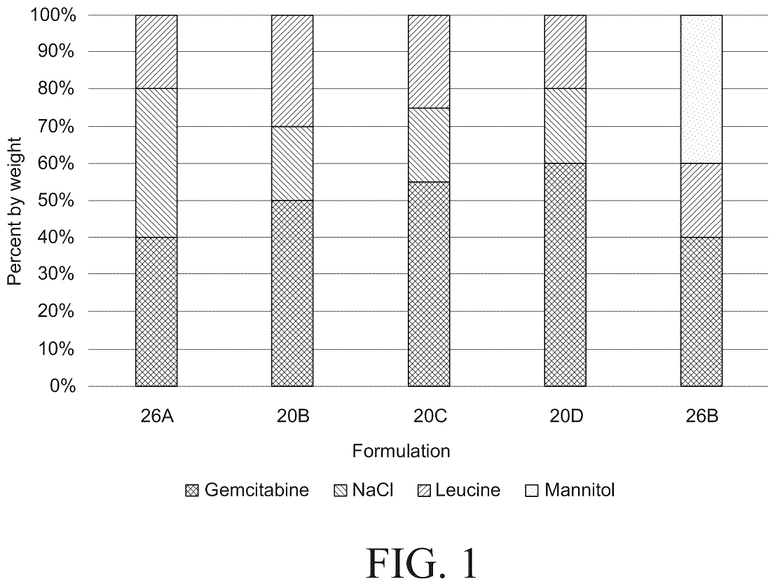

In this example, pharmaceutical formulations were identified for production and testing. Five dry powder pharmaceutical formulations (20B, 20C, 20D, 26A and 26B) were calculated for optimal targeted delivery of an API of gemcitabine to the lung (e.g., the small airways, which are typically the 8^{th}-18^{th} branches of the lung). The pharmaceutical formulations included sodium chloride (NaCl) or mannitol (MN) as a growth enhancing excipient and the gemcitabine-to-growth enhancing excipient ratio was varied. Anatomically relevant computational fluid dynamic (CFD) modeling data was analyzed in combination with realistic airway in-vitro experimental data to identify formulation gemcitabine-to-excipient ratios with a target initial particle size of 1.0-2.0 μm volume median diameter (a critical quality attribute) to achieve a target growth ratio of 2-4 within the airways. These formulation properties may avoid mouth-throat deposition and enable high efficiency deposition and retention in the small airways, respectively. The pharmaceutical formulations further included 20% or greater leucine as a dispersion enhancing agent. This provided good dispersion of the powders in an initial powder mass of 20 mg with particle densities ranging from 1.55 g/cm³-1.72 g/cm³. The amount of gemcitabine was targeted between 40-60% of the pharmaceutical formulations for delivery of a 20 mg powder load in size 3 capsules, thus minimizing the number of capsules required per treatment while maintaining proper loading and unloading of the dry powder formulation in the capsules. The compositions of the five pharmaceutical formulations with the target drug-excipient ratios are shown in FIG. 1. Referring now to FIG. 1, the graph shows that formulation 20B included 50% gemcitabine, 20% sodium chloride (NaCl), and 30% leucine, formulation 20C included 55% gemcitabine, 20% sodium chloride (NaCl), and 25% leucine, formulation 20D included 60% gemcitabine, 20% sodium chloride (NaCl), and 20% leucine, formulation 26A included 40% gemcitabine, 40% sodium chloride (NaCl), and 20% leucine, and formulation 26B included 40% gemcitabine, 20% mannitol, and 20% leucine, all by weight. These results demonstrate that five dry powder formulations, and desired initial particle size, growth ratio, and final particle size targets for the manufacturing production, were identified from analysis of modeling and experimental data. The pharmaceutical formulations included gemcitabine as an active pharmaceutical ingredient, leucine as a dispersion enhancing agent, and either mannitol (MN) or sodium chloride (NaCl) as a growth enhancing excipient, and were optimized to achieve maximum lung penetration and deposition in the small airways.

### Example 2

### Manufacture of Gemcitabine Pharmaceutical Formulations

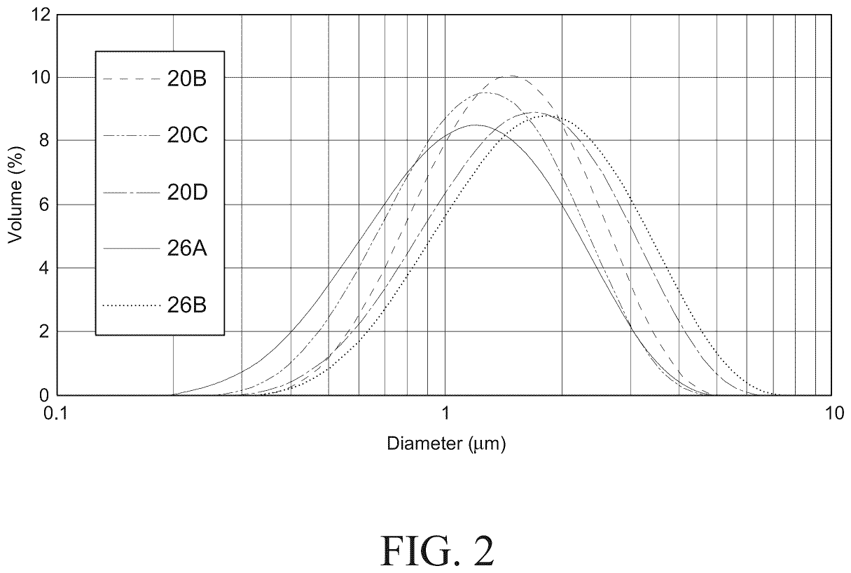

In this example, the five dry powder pharmaceutical formulations identified in FIG. 1 (20B, 20C, 20D, 26A and 26B) were manufactured by spray drying. The formulations were manufactured on a lab scale dryer with 35 kg/hr drying gas capacity (BLD-35). The spray drying process included cyclone collection that was optimized for the critical quality attribute of particle size. The spray drying and cyclone collection process was customized and optimized to achieve the required formulation particle size using the desired gemcitabine to excipient ratios. Briefly, a stock solution containing 1% total solids in distilled water was used as the spray vehicle. The feed rate range was 6-7 mL/min with atomization pressures in the range of 25-60 psi. The inlet temperature range was 123-129° C. with an outlet temperature of 60° C. In-process particle size testing throughout the batch manufacture was conducted to ensure the particle size specifications were being met throughout the manufacturing process. An initial characterization of the formulations included the following tests: water content by KF, assay/RS by HPLC, particle morphology by SEM, thermal characterization by mDSC, appearance, physical state by XRPD, particle size distribution by Malvern, and water sorption by DVS. The results of the initial characterization are shown in Table 1. The particle size distributions of the dry powder pharmaceutical formulations manufactured by spray drying are shown in FIG. 2.

TABLE 1
Initial Analytical Summary

| | BREC-0984- | BREC-0984- | BREC-0984- | BREC-0984- | BREC-0984- |
|---|---|---|---|---|---|
| Lot Number | 026A | 020B | 020C | 020D | 026B |
| Formulation | 40/40/20% | 50/20/30% | 55/20/25% | 60/20/20% | 40/40/20% |
| | Gem/NaCl/ | Gem/NaCl/ | Gem/NaCl/ | Gem/NaCl/ | Gem/Mannitol/ |
| | Leucine | Leucine | Leucine | Leucine | Leucine |
| KF (wt. %) | 2.04 ± 0.03 | 2.14 ± 0.02 | 2.43 ± 0.00 | 2.70 ± 0.06 | 1.53 ± 0.01 |
| Assay (mgA/g) | 373 ± 1.2 | 488.2 ± 0.7 | 459.1 ± 17.1 | 598.0 ± 4.1 | 345.5 ± 34.2 |
| | (93.3%) | (97.6%) | (83.5%) | (99.7%) | (86.4%) |
| SEM | Spherical | Collapsed | Collapsed | Spherical | Smooth |
| | particles, no | spherical | spherical | particles, no | spherical |
| | evidence of | particles, no | particles, no | evidence of | particles, no |
| | fusion or | evidence of | evidence of | fusion or | evidence of |
| | surface | fusion or | fusion or | surface | fusion or |
| | crystallization | surface | surface | crystallization | surface |
| | | crystallization | crystallization | | crystallization |
| mDSC (Tg) ° C. | 98.0 ± 0.4 | 98.1 ± 0.3 | 99.2 ± 0.5 | 100.2 ± 0.4 | 70.6 ± 0.7 |
| DVS | Potential | — | — | — | Potential |
| | crystallization | | | | crystallization |
| | event starting | | | | event starting |
| | 40% RH | | | | 40% RH |
| Appearance | Homogenous, white powder, free of foreign material with some visible clumps | | | | |
| XRPD | Presence of | Presence of | Presence of | Presence of | Presence of |
| | crystalline | crystalline | crystalline | crystalline | crystalline |
| | excipient | excipient | excipient | excipient | excipient |
| | peaks, | peaks, | peaks, | peaks, | peaks, |
| | suggesting | suggesting | suggesting | suggesting | suggesting |
| | amorphous API | amorphous API | amorphous API | amorphous API | amorphous API |
| Target D50 | 1.3 | 1.6 | 1.4 | 1.8 | 1.9 |
| (μm) | | | | | |
| Measured | 0.6/1.2/2.5 | 0.8/1.5/2.8 | 0.7/1.3/2.5 | 0.8/1.7/3.3 | 0.9/1.9/3.7 |
| PSD (μm) | | | | | |

Referring now to FIG. 2, the graph shows particle size distributions of the five dry powder pharmaceutical formulations manufactured in 8 g batch sizes. The particle size distribution was measured using Sympatec laser diffraction. Observed initial particle sizes for the formulations were 1.0-2.0 μm volume median diameter. These values were targeted for high efficiency aerosol performance combined with efficient manufacturing. Powder yields were between 70-90% for all formulations. These results demonstrate that efficient production of micrometer-sized gemcitabine dry powder formulations of the present disclosure can be achieved by spray drying.

### Example 3

### Characterization, Stability, and Aerosol Performance Testing of Gemcitabine Pharmaceutical Formulations

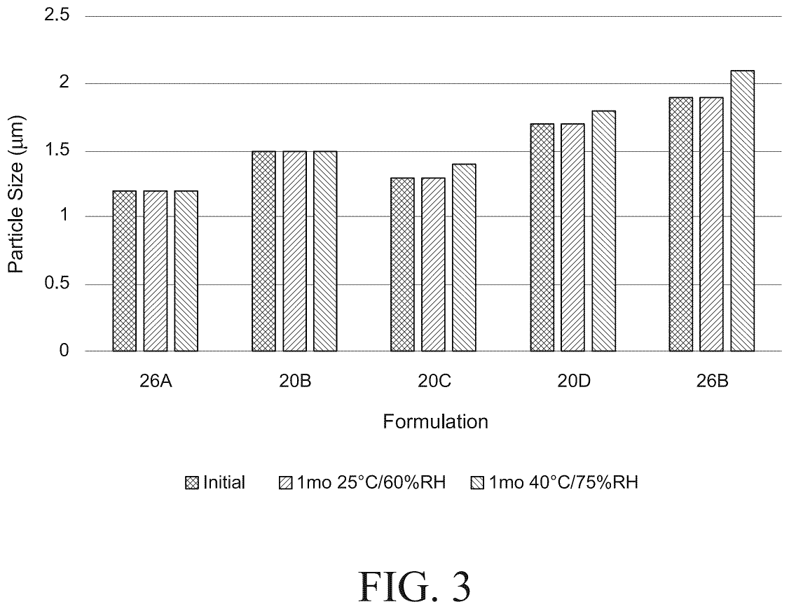

In this example, the pharmaceutical feasibility for pharmaceutical formulations 20B, 20C, 20D, 26A and 26B was investigated. The particle size distribution and aerosol performance were measured directly after initial production and then after a 1-month period. The particle size distribution was characterized by laser diffraction. Other characterization studies included water and solvent content (e.g., as measured by Karl-Fisher and thermogravimetric weight loss), crystallinity and amorphous content (e.g., as measured by differential scanning calorimetry and/or powder x-ray diffraction), dynamic vapor sorption, appearance by scanning electron microscope (e.g., as measured by SEM). Stability studies were performed on powder formulations loaded into standard hydroxypropyl methylcellulose (HPMC) capsules in primary packaging with the addition of a secondary packaging pouch. The particle size characteristics (volume median diameter) of the powder formulations tested under ambient (25° C./60% RH) and accelerated conditions (40° C./75% RH) at the 1-month time point are shown in FIG. 3. Referring now to FIG. 3, the graph shows that the formulation particle size distributions do not change significantly in the month after initial production when stored at ambient and accelerated conditions and are within the desired specifications for the pharmaceutical formulations of the present disclosure to reach the small airways when administered by aerosol delivery.

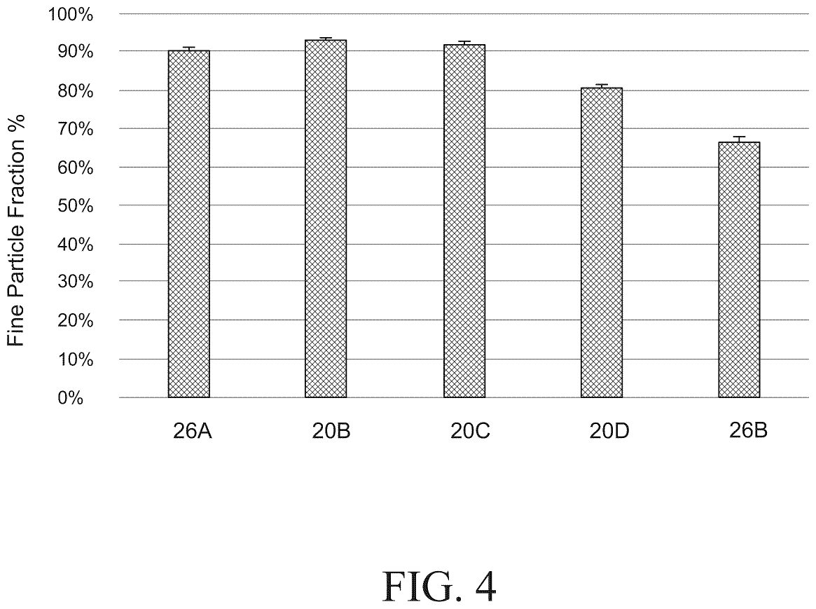

Fine particle fractions (FPF) were measured for the five pharmaceutical formulations one month after initial production using a standard rapid impactor method. The Plastiape RS01 single capsule delivery device (dry powder inhaler (DPI)) was used to deliver the pharmaceutical formulations. FIG. 4 shows the fine particle fraction (FPF), measured as % particles less than 3.5 μm expressed as a percentage of the emitted dose following storage for one month at ambient conditions (25° C./60% RH). Referring now to FIG. 4, the graph shows that three of the five pharmaceutical formulations have fine particle fractions of 90% or greater. The formulation 20B had a fine particle fraction of 93%. These results demonstrate that 1 month after initial production, the pharmaceutical formulations of the present disclosure performed well, even using a commercial DPI (the RS01 DPI) not specifically designed for the pharmaceutical formulations.

### Example 4

### In-Vivo Efficacy of Inhaled Gemcitabine Pharmaceutical Formulations

In this example, the in-vivo efficacy of pharmaceutical formulation 20B was tested. Pharmaceutical formulation 20B includes sodium chloride as the growth enhancing excipient. In particular, pharmaceutical formulation 20B includes 50% gemcitabine, 20% NaCl, and 30% leucine, by weight. Pharmaceutical formulation 20B was selected from the aggregate characterization, stability, and aerosol performance data as a lead formulation. Pharmaceutical formulation 20B is characterized by a volume median diameter of 1.5 μm, low polydispersity (FIG. 2), highest fine particle fraction (FIG. 4) and its primary particle size was unaltered following 1-month storage at ambient and accelerated conditions (FIG. 3). For pre-clinical in-vivo efficacy testing, a 24 g batch of pharmaceutical formulation 20B was manufactured using the same conditions as described in Example 2.

A pre-clinical in-vivo inhalation efficacy study was conducted in a lung cancer rodent model. A well-established orthotopic lung cancer rodent model in Rowett nude rats was utilized for this efficacy study that was developed by Lovelace Institute (March, T. H., P. G. Marron-Terada, and S. A. Belinsky, Vet Pathol., 2001. 38(5):483-490). The efficacy study was designed with 4 tumor arms (3 with treatment, one with no treatment) and one naïve arm (no tumor cells, no treatment) of 15 rodents in each arm, as shown in Table 2.

TABLE 2
In-vivo Efficacy Study Design

| | Tumor | Treatment | | |
|---|---|---|---|---|
| Arm | Model | Route | Dose | |
| Naive Control | No | NA | NA | |
| Sham Control | Yes | NA | NA | |
| Standard of Care-IV | Yes | Intravenous | 1 | mg/kg |
| Inhalation-IV dose | Yes | Inhalation | 1 | mg/kg |
| Inhalation-1/2 IV dose | Yes | Inhalation | 0.5 | mg/kg |

The tumor arms of the efficacy study were as follows: “untreated” with no treatment (sham control), intravenous (IV) standard of care treatment with 1.0 mg/kg gemcitabine and two treatment arms with an inhaled pharmaceutical formulation 20B with doses of 1.0 mg/kg and 0.5 mg/kg gemcitabine. Inhalation doses were based on previous in-vivo aerosolized gemcitabine work conducted at MD Anderson Cancer Center (Texas) and were designed to examine the comparison of the standard clinical IV dose to inhalation doses of the same dose (1.0 mg/kg) and a lower dose (0.5 mg/kg).

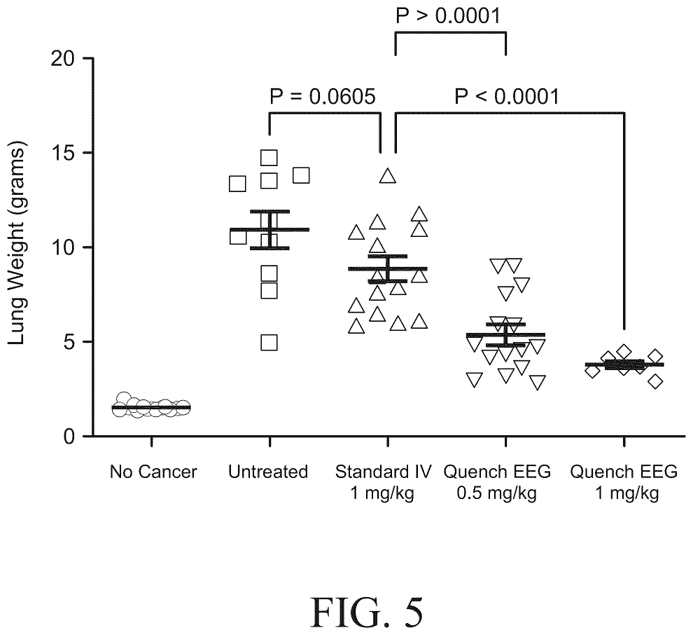

On day 0, human lung adenocarcinoma A549 tumor cells (15×10⁶ cells/rat) were implanted into the rat lungs via the trachea. Starting on day 25, three weeks after tumor establishment and growth, the animals were treated once-a-week for 4 consecutive weeks and were sacrificed for moribund conditions or at the end of the study on day 54. The lungs from all animals were excised and weighed with tracheas attached. The primary endpoint of efficacy was tumor burden, as measured by lung weight. FIG. 5 shows the results of these experiments. Referring now to FIG. 5, the graph shows that both pharmaceutical formulation 20B aerosol treatment groups significantly decreased the tumor burden compared to standard of care IV. Both doses of pharmaceutical formulation 20B were significantly more effective compared to the intravenous (IV) standard of care group (P<0.0001) and untreated control group (P<0.0001). It has been demonstrated that treatment-related reduction in tumor burden is highly correlated with estimates of tumor volume.

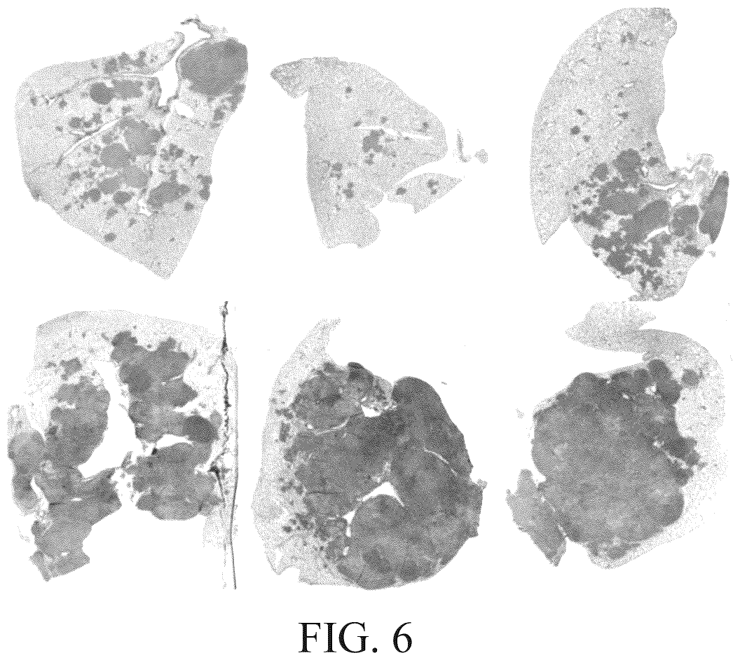

Therapeutic response was also assessed by histologic analysis of lung samples from the different groups for evidence of micro-metastases. Samples were fixed, processed, and embedded in paraffin. Hematoxylin-Eosin (H&E) staining was performed and demonstrated various degrees of tumor burden as assessed by number and size of micro-metastases. Micro-metastatic lesions were assessed by light microscopy using various magnifications (10×, 20× and 40×) and identified as infiltrating lesions within the lung parenchyma with disrupted cytoplasm and increase in cell mitosis. FIG. 6 shows the results of the histologic analysis. Referring now to FIG. 6, FIG. 6 shows a significant decrease in tumor burden with the inhaled pharmaceutical formulation 20B therapy at half of the dose (top row) compared to intravenous standard of care (bottom row). These results demonstrate that pharmaceutical formulation 20B is highly successful in reducing tumor burden compared to standard of care, even at half of the dose.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference in their entirety. Aspects of the embodiments may be modified, if necessary, to employ concepts of the various patents, applications and publications to provide further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A pharmaceutical formulation, comprising:
at least one active pharmaceutical ingredient (API) or a pharmaceutically acceptable salt, ester, derivative, analog, prodrug, hydrate, or solvate thereof, or any combination thereof;
at least one growth enhancing excipient; and
at least one dispersion enhancing agent;
wherein the pharmaceutical formulation is a dry powder having a particle size of from about 0.5 μm to about 2.5 μm volume median diameter.

2. The pharmaceutical formulation of claim 1, wherein the API comprises a chemotherapeutic agent.

3. The pharmaceutical formulation of claim 2, wherein the chemotherapeutic agent is gemcitabine.

4. The pharmaceutical formulation of any one of claims 1-3, wherein the growth enhancing excipient comprises sodium chloride (NaCl), mannitol, or a combination thereof.

5. The pharmaceutical formulation of any one of claims 1-4, wherein the dispersion enhancing agent comprises leucine.

6. The pharmaceutical formulation of any one of claims 1-5, wherein the pharmaceutical formulation comprises the API in an amount from about 30% to about 70% by weight of the formulation.

7. The pharmaceutical formulation of any one of claims 1-6, wherein the pharmaceutical formulation comprises the growth enhancing excipient in an amount from about 15% to about 50% by weight of the formulation.

8. The pharmaceutical formulation of any one of claims 1-7, wherein the pharmaceutical formulation comprises the dispersion enhancing agent in an amount from about 15% to about 45% by weight of the formulation.

9. The pharmaceutical formulation of any one of claims 1-5, wherein the pharmaceutical formulation comprises:
the API in an amount of about 50% by weight of the formulation;
the growth enhancing excipient in an amount of about 20% by weight of the formulation; and
the dispersion enhancing agent in an amount of about 30% by weight of the formulation.

10. The pharmaceutical formulation of any one of claims 1-5, wherein the pharmaceutical formulation comprises:
the API in an amount of about 40% by weight of the formulation;
the growth enhancing excipient in an amount of about 40% by weight of the formulation; and
the dispersion enhancing agent in an amount of about 20% by weight of the formulation.

11. The pharmaceutical formulation of any one of claims 1-5, wherein the pharmaceutical formulation comprises:
the API in an amount of about 55% by weight of the formulation;
the growth enhancing excipient in an amount of about 20% by weight of the formulation; and
the dispersion enhancing agent in an amount of about 25% by weight of the formulation.

12. The pharmaceutical formulation of any one of claims 1-5, wherein the pharmaceutical formulation comprises:
the API in an amount of about 60% by weight of the formulation;
the growth enhancing excipient in an amount of about 20% by weight of the formulation; and
the dispersion enhancing agent in an amount of about 20% by weight of the formulation.

13. The pharmaceutical formulation of any one of claims 1-5, wherein the pharmaceutical formulation comprises:
the API in an amount of about 40% by weight of the formulation;
the growth enhancing excipient in an amount of about 20% by weight of the formulation; and
the dispersion enhancing agent in an amount of about 40% by weight of the formulation.

14. The pharmaceutical formulation of any one of claims 1-13, wherein the particle size is from about 1 μm to about 2.1 μm volume median diameter.

15. The pharmaceutical formulation of any one of claims 1-13, wherein the particle size is about 1.5 μm volume median diameter.

16. The pharmaceutical formulation of any one of claims 1-13, wherein the particle size is about 1.2 μm volume median diameter.

17. The pharmaceutical formulation of any one of claims 1-13, wherein the particle size is about 1.3 μm volume median diameter.

18. The pharmaceutical formulation of any one of claims 1-13, wherein the particle size is about 1.7 μm volume median diameter.

19. The pharmaceutical formulation of any one of claims 1-13, wherein the particle size is about 1.9 μm volume median diameter.

20. The pharmaceutical formulation of any one of claims 1-13, wherein a particle size distribution has a D10 of about 0.6 μm, a D50 of about 1.2 μm, and a D90 of about 2.5 μm.

21. The pharmaceutical formulation of any one of claims 1-13, wherein a particle size distribution has a D10 of about 0.8 μm, a D50 of about 1.5 μm, and a D90 of about 2.8 μm.

22. The pharmaceutical formulation of any one of claims 1-13, wherein a particle size distribution has a D10 of about 0.7 μm, a D50 of about 1.3 μm, and a D90 of about 2.5 μm.

23. The pharmaceutical formulation of any one of claims 1-13, wherein a particle size distribution has a D10 of about 0.8 μm, a D50 of about 1.7 μm, and a D90 of about 3.3 μm.

24. The pharmaceutical formulation of any one of claims 1-13, wherein a particle size distribution has a D10 of about 0.9 μm, a D50 of about 1.9 μm, and a D90 of about 3.7 μm.

25. The pharmaceutical formulation of any one of claims 1-24, wherein the pharmaceutical formulation is packaged in a capsule or a blister pack.

26. The pharmaceutical formulation of any one of claims 1-25, wherein the pharmaceutical formulation is manufactured by spray drying.

27. The pharmaceutical formulation of any one of claims 1-26, wherein the volume median diameter does not change by more than 10% following storage for at least one month at 25° C. (77° F.) and 60% relative humidity.

28. The pharmaceutical formulation of any one of claims 1-26, wherein the volume median diameter does not change by more than 20% following storage for at least one month at 40° C. (104° F.) and 75% relative humidity.

29. The pharmaceutical formulation of any one of claims 1-28, wherein the pharmaceutical formulation is formulated such that the particle size of at least 60% of particles is less than about 3.5 μm volume median diameter when the pharmaceutical formulation is administered using a dry powder inhaler following storage for at least one month at 25° C. (77° F.) and 60% relative humidity.

30. The pharmaceutical formulation of any one of claims 1-29, wherein the pharmaceutical formulation is formulated for administration by aerosol delivery.

31. The pharmaceutical formulation of claim 30, wherein the pharmaceutical formulation is formulated for administration using a dry powder inhaler.

32. The pharmaceutical formulation of claim 31, wherein the pharmaceutical formulation is packaged into a single capsule for administration using the dry powder inhaler.

33. A method of treating lung cancer, comprising:
administering to a lung of a subject in need thereof of a therapeutically effective amount of a pharmaceutical formulation of any one of claims 1-32, wherein the pharmaceutical formulation is administered as a dry powder aerosol.

34. The method of claim 33, wherein the pharmaceutical formulation is deposited in the lung in an amount of at least about 50% by weight of the pharmaceutical formulation.

35. The method of claim 33, wherein the pharmaceutical formulation is deposited in the lung in an amount of at least about 80% by weight of the pharmaceutical formulation.

36. The method of any one of claims 33-35, wherein the pharmaceutical formulation is deposited in the throat, mouth, or a combination thereof, in an amount of less than about 20% by weight of the pharmaceutical formulation.

37. The method of any one of claims 33-36, wherein the subject has a suboptimal or inadequate response to intravenous delivery of the API.

38. The method of any one of claims 33-37, wherein administering the pharmaceutical formulation results in a reduced tumor burden as compared to intravenous delivery of an equivalent dose of the API.

39. The method of any one of claims 33-38, wherein the lung cancer is a secondary lung cancer.

40. A method of delivery of a chemotherapeutic agent to a lung of a subject in need thereof, comprising:
administering a pharmaceutical formulation, comprising:
at least one chemotherapeutic agent;
at least one growth enhancing excipient; and
at least one dispersion enhancing agent;
wherein the pharmaceutical formulation is a dry powder having a particle size of from about 0.5 μm to about 2.5 μm volume median diameter, the pharmaceutical formulation is administered using a dry powder inhaler, and the pharmaceutical formulation is deposited in the lung in an amount of at least about 50% by weight of the pharmaceutical formulation.

41. The method of claim 40, wherein the chemotherapeutic agent is gemcitabine, or a pharmaceutically acceptable salt, ester, derivative, analog, prodrug, hydrate, or solvate thereof, or any combination thereof.

42. The method of any one of claims 40 and 41, wherein the growth enhancing excipient comprises sodium chloride (NaCl), mannitol, or a combination thereof.

43. The method of any one of claims 40-42, wherein the dispersion enhancing agent comprises leucine.

44. The method of any one of claims 40-43, wherein the pharmaceutical formulation comprises the chemotherapeutic agent in an amount from about 30% to about 70% by weight of the formulation.

45. The method of any one of claims 40-44, wherein the pharmaceutical formulation comprises the growth enhancing excipient in an amount from about 15% to about 50% by weight of the formulation.

46. The method of any one of claims 40-45, wherein the pharmaceutical formulation comprises the dispersion enhancing agent in an amount from about 15% to about 45% by weight of the formulation.

47. The method of any one of claims 40-43, wherein the pharmaceutical formulation comprises:
the chemotherapeutic agent in an amount of about 50% by weight of the formulation;
the growth enhancing excipient in an amount of about 20% by weight of the formulation; and
the dispersion enhancing agent in an amount of about 30% by weight of the formulation.

48. The method of any one of claims 40-43, wherein the pharmaceutical formulation comprises:
the chemotherapeutic agent in an amount of about 40% by weight of the formulation;
the growth enhancing excipient in an amount of about 40% by weight of the formulation; and
the dispersion enhancing agent in an amount of about 20% by weight of the formulation.

49. The method of any one of claims 40-43, wherein the pharmaceutical formulation comprises:
the chemotherapeutic agent in an amount of about 55% by weight of the formulation;
the growth enhancing excipient in an amount of about 20% by weight of the formulation; and
the dispersion enhancing agent in an amount of about 25% by weight of the formulation.

50. The method of any one of claims 40-43, wherein the pharmaceutical formulation comprises:
the chemotherapeutic agent in an amount of about 60% by weight of the formulation;
the growth enhancing excipient in an amount of about 20% by weight of the formulation; and
the dispersion enhancing agent in an amount of about 20% by weight of the formulation.

51. The method of any one of claims 40-43, wherein the pharmaceutical formulation comprises:
the chemotherapeutic agent in an amount of about 40% by weight of the formulation;
the growth enhancing excipient in an amount of about 20% by weight of the formulation; and
the dispersion enhancing agent in an amount of about 40% by weight of the formulation.

52. The method of any one of claims 40-51, wherein the particle size is from about 1 μm to about 2.1 μm volume median diameter.

53. The method of any one of claims 40-51, wherein the particle size is about 1.5 μm volume median diameter.

54. The method of any one of claims 40-51, wherein the particle size is about 1.2 μm volume median diameter.

55. The method of any one of claims 40-51, wherein the particle size is about 1.3 μm volume median diameter.

56. The method of any one of claims 40-51, wherein the particle size is about 1.7 μm volume median diameter.

57. The method of any one of claims 40-51, wherein the particle size is about 1.9 μm volume median diameter.

58. The method of any one of claims 40-51, wherein a particle size distribution has a D10 of about 0.6 μm, a D50 of about 1.2 μm, and a D90 of about 2.5 μm.

59. The method of any one of claims 40-51, wherein a particle size distribution has a D10 of about 0.8 μm, a D50 of about 1.5 μm, and a D90 of about 2.8 μm.

60. The method of any one of claims 40-51, wherein a particle size distribution has a D10 of about 0.7 μm, a D50 of about 1.3 μm, and a D90 of about 2.5 μm.

61. The method of any one of claims 40-51, wherein a particle size distribution has a D10 of about 0.8 μm, a D50 of about 1.7 μm, and a D90 of about 3.3 μm.

62. The method of any one of claims 40-51, wherein a particle size distribution has a D10 of about 0.9 μm, a D50 of about 1.9 μm, and a D90 of about 3.7 μm.

63. The method of any one of claims 40-62, wherein the pharmaceutical formulation is packaged in a capsule or a blister pack.

64. The method of any one of claims 40-63, wherein the pharmaceutical formulation is manufactured by spray drying.

65. The method of any one of claims 40-64, wherein the volume median diameter of the pharmaceutical formulation does not change by more than 10% following storage for at least one month at 25° C. (77° F.) and 60% relative humidity.

66. The method of any one of claims 40-64, wherein the volume median diameter of the pharmaceutical formulation does not change by more than 20% following storage for at least one month at 40° C. (104° F.) and 75% relative humidity.

67. The method of any one of claims 40-66, wherein the pharmaceutical formulation is formulated such that the particle size of at least 60% of particles is less than about 3.5 μm volume median diameter when the pharmaceutical formulation is administered using a dry powder inhaler following storage for at least one month at 25° C. (77° F.) and 60% relative humidity.

68. The method of claims 40-67, wherein the pharmaceutical formulation is packaged into a single capsule for administration using the dry powder inhaler.

69. Use of the pharmaceutical formulation of any one of claims 1-32 in the manufacture of a medicament for the treatment of lung cancer, wherein the pharmaceutical formulation is to be administered to a lung of a subject as a dry powder aerosol.

70. The pharmaceutical formulation of any one of claims 1-32 for use in the treatment of lung cancer in a subject, wherein the pharmaceutical formulation is to be administered to a lung of the subject as a dry powder aerosol.

71. Use of the pharmaceutical formulation of any one of claims 1-32 for treating lung cancer in a subject, wherein the pharmaceutical formulation is for administration to a lung of the subject as a dry powder aerosol.
